(19) United States
(12) Patent Application Publication
(10) Pub. No.: US 2026/0240456 A1
(43) Pub. Date: Aug. 20, 2026

(54) MULTIMODAL ULTRASOUND AND ACOUSTIC SYSTEM AND METHOD FOR NOVEL NON-INVASIVE HEARING ASSESSMENT AND INTRACRANIAL PRESSURE MONITORING

(71) Applicant: Regents of the University of Minnesota, Minneapolis, MN (US)

(72) Inventors: Hubert H. LIM, Minneapolis, MN (US); Meredith E. ADAMS, Minneapolis, MN (US); Tyler GATHMAN, Minneapolis, MN (US); Gerardo RODRIGUEZ-ORELLANA, Minneapolis, MN (US); John BASILE, Minneapolis, MN (US)

(21) Appl. No.: 19/153,797

(22) PCT Filed: Feb. 9, 2024
(86) PCT No.: PCT/US2024/015198

§ 371 Date: Aug. 5, 2025

Related U.S. Application Data
(60) Provisional application No. 63/444,646, filed on Feb. 10, 2023.

(51) Int. Cl. A61B 5/12; A61B 5/38

(52) U.S. Cl. CPC A61B 5/125; A61B 5/38

(57) ABSTRACT

An apparatus for noninvasive assessment of at least one of hearing or intracranial pressure, including: an ultrasound transducer configured to contact a head or a body of a subject; a sound generator; a microphone configured to measure an auditory response; and a controller operatively coupled to the ultrasound system, the sound generator, and the microphone and configured to: transmit an ultrasound wave pattern using the ultrasound transducer, produce a sound signal using the sound generator, measure an auditory response using the microphone, and analyze the auditory response to estimate at least one of hearing function or intracranial pressure of the subject.

### RELATED APPLICATIONS

This application is based on, claims priority to, and incorporates herein by reference for all purposes, U.S. Provisional Patent Application No. 63/444,646, filed on Feb. 10, 2023.

### STATEMENT REGARDING FEDERALLY SPONSORED RESEARCH

N/A

### BACKGROUND

Hearing loss is a widespread and morbid condition that affects individuals of all ages from congenital to adult-onset impairment. Additionally, intracranial hypertension, or elevated intracranial pressure (ICP), remains difficult to diagnosis and can lead to neurological impairment if not promptly identified.

### SUMMARY

Some embodiments of the invention provide an apparatus for noninvasive assessment of hearing or intracranial pressure. The apparatus may include an ultrasound transducer that can be configured to be placed on the head or body of a subject and transmit ultrasound waves of various frequencies, amplitudes, patterns, etc. The apparatus further includes a sound generator to produce an audio signal and a microphone to measure an auditory response. The apparatus further includes a controller that is operatively coupled to the ultrasound transducer, sound generator, and microphone. The controller may control the ultrasound transducer to transmit an ultrasound wave pattern. The controller may also control the sound generator to produce a sound signal and control the microphone to measure an auditory response signal. The controller may also be configured to analyze the auditory response to estimate hearing or intracranial pressure. The controller may also be configured to analyze the auditory response along with optical images or a neural response to estimate hearing or intracranial pressure.

Some embodiments of the invention provide a method for noninvasive assessment of hearing or intracranial pressure. The method may include providing an apparatus, which includes an ultrasound transducer that is configured to contact a head or body of a subject, a sound generator, a microphone that is configured to measure an auditory response, and a controller that is operatively coupled to the ultrasound transducer, sound generator, and microphone. The method further includes producing a sound signal using the sound generator and measuring an auditory response using the microphone. The method can analyze the auditory response to estimate at least one of hearing function or intracranial pressure of the subject. The method may also analyze the auditory response with optical images or a neural response to estimate at least one of hearing function or intracranial pressure of the subject.

### DESCRIPTION OF THE DRAWINGS

FIG. 1 provides an overview of human ear anatomy. The cochlea and contained Organ of Corti enable transduction of mechanical fluid vibrations to neural signals within the Auditory nerve.

FIG. 2 shows that the Organ of Corti within the cochlea produces neural signals in the Auditory nerve from inner hair cells in response to fluid vibrations within the scala tympani that are transmitted to the Basilar membrane.

FIG. 3 illustrates how otoacoustic emissions, including DPOAEs are generated by OHCs within the cochlea and travel retrograde from the inner to middle ear and then to the ear canal where external microphones can measure the response.

FIG. 4 illustrates clinical DPOAE in response to a stimulation (f₁ and f₂) due to non-linear amplification (i.e., cochlea amplifier) of the outer hair cells.

FIG. 5 shows example MRI images demonstrating non-communication hydrocephalus with excessive CSF infiltrating the ventricles leading the elevated ICP and consequently compression of neurological structures.

FIG. 6 is a block diagram that illustrates the components of the multimodal ultrasound and acoustic system according to some embodiments of the invention.

FIG. 7A is a flow chart that illustrates methods for use with the multimodal ultrasound and acoustic system according to some implementations of the invention.

FIG. 7B shows a schematic of an example multimodal ultrasound and acoustic system as it can interact with a physiological system according to some aspects of the present disclosure.

FIG. 8A is an example ear-bar, which can be used in accordance with some embodiments of the invention.

FIG. 8B is an experimental schematic of hydrostatic elevation of ICP in an animal model including continuous monitoring of DPOAEs and US or acoustic stimulation.

FIG. 9 shows that, with a high enough sampling rate (e.g., 1 kHz), the ICP waveform can be appreciated with a frequency of approximately 200 cycles per minute consistent with the normal heart rate of the animal model.

FIG. 10A shows representative DPOAE spectra for acoustic, US-evoked, and bimodal modalities with acoustic levels of 60-dB SPL and US levels of 70 kPa.

FIG. 10B shows representative spectra for control experiments in which the acoustic speaker or ultrasound transducer is removed.

FIG. 11 shows DPOAE spectra before and 60 minutes after euthanization, which show prominent decrease in signal amplitude.

FIG. 12 show the temporal trend of DPOAE amplitude after administration of euthanization, characterized by prominent down sloping consistent with decrease in OHC and cochlear activity.

FIG. 13 shows a stability plot over 20 minutes for all modalities, which reflects time-invariancy and reliability of the experimental setup.

FIG. 14 shows that ICP was increased step-wise for three levels with 15 minutes at each pressure to measure DPOAE responses.

FIG. 15 shows how DPOAE amplitude remained approximately stable with moderate ICP elevations but dropped significantly at severely increased pressures.

FIG. 16A shows example experimental results of a DPOAE evoked from acoustic stimuli.

FIG. 16B shows example experimental results of a DPOAE evoked from bimodal stimuli.

FIG. 17A shows example experimental results of acoustic-evoked DPOAEs before and after ultrasound stimulation.

FIG. 17B shows example experimental results of bimodal-evoked DPOAEs over varying ultrasound power.

FIG. 17C shows example experimental results of ultrasound evoked DPOAEs over varying ultrasound power.

FIG. 18A shows example experimental results showing baseline measure of acoustic evoked DPOAEs.

FIG. 18B shows example experimental results of bimodal evoked DPOAEs.

FIG. 18C shows example experimental results of ultrasound evoked DPOAEs.

FIG. 18D shows example experimental results of acoustic evoked DPOAEs after exposure to an ultrasound stimulus.

FIG. 19 shows a schematic representation of the fluid pathway by which ultrasound activates the auditory system.

FIG. 20A shows a schematic representation of an experimental setup according to some aspects of the present disclosure.

FIG. 20B shows an example use of earth mover's distance representation to measure distance between histograms according to some aspects of the present disclosure.

FIG. 21 shows sample frequency response maps and ultrasound-frequency response maps. Channels have been sorted so that the top-most is the lowest frequency and the bottom-most is the highest frequency. Ultrasound intensities ranged from 7-70 kPa, and acoustic intensities ranged from 0-70 dB SPL. The US center frequency was 220 kHz with modulation of 1-40 kHz tones. These tones were used create the acoustic FRM.

FIG. 22 plots the best frequency of all channels in response to ultrasound and acoustic stimulation. The “best” frequency (BF) is the modulation or tone that evoked activity at the lowest sound or pressure intensity. As seen, the ultrasound and acoustic BF lie close to the y=x axis, showing the envelope of ultrasound encodes the acoustic information reliably.

FIG. 23 shows example experimental results of population response maps evoked by varying stimulation types and signals.

FIG. 24A shows PSTHs of a representative channel in response to different stimuli. The top row traces demonstrate responses to ultrasound, while the bottom row traces demonstrate responses to air-driven stimuli. Each PSTH evoked by ultrasound was compared to those evoked by air-driven stimuli. When the comparison was between different stimuli (i.e., Chutter-1 vs Scream-1, black lines), the distances were aggregated and classified as Different Stimulus. When the comparison was between similar stimuli (i.e., Chutter-1 vs Chutter-1, grey line), the distances were aggregated and classified as Same Stimulus. Red lines across the bottoms of the histograms highlight the duration of the stimulus.

FIG. 24B shows EMD totaled across all active channels. Error bars+/−1 SEM. T-test comparison between same and different stimuli (p<0.01) adjusted for multiple comparisons.

FIG. 24C shows EMD of ultrasound stimulation and air-driven stimulation across frequency bands.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

The present disclosure describes an apparatus and method for non-invasively assessing hearing and ICP using ultrasound to activate the auditory system. The ultrasound-based apparatus may be used to assess hearing function that can identify features not possible or complementary to current traditional audio-based methods. The apparatus may also be used to assess intracranial pressure based on the acoustic or auditory response by using a model that relates the acoustic or auditory response to an intracranial pressure. The microphones or speakers may be placed in the ears of the subject. The microphones may be configured to record otoacoustic emissions or distortion production otoacoustic emissions. The apparatus may further include one or more electrodes that can measure a neural response to the stimuli.

Current hearing assessment methods, based on auditory stimulation by sound, do not always provide comprehensive or complete characterization of hearing ability and thus may miss individuals with hearing loss or some compromise of hearing function that are not picked up by current diagnostic techniques. Ultrasound activates the cochlea and auditory system in different or overlapping ways than sound stimulation. A novel US-based hearing assessment method would provide additional, complimentary information that may more accurately detect hearing impairment in a variety of populations and hearing conditions. Additionally, the standard diagnosis for intracranial pressure (ICP) includes use of a lumbar puncture or intraventricular catheters, which are invasive, expensive, painful, and fear-provoking in patients. Moreover, such invasive methods can lead to infection, hemorrhage, neuropathies, or other chronic complications. Thus, a non-invasive method of measuring ICP could minimize complications and patient anxiety, while providing rapid results and early diagnosis, thus improving prognosis. The systems and methods described herein provide a non-invasive method of measuring intracranial pressure and identifying intracranial hypertension, which could expedite the diagnosis of brain/spinal pressure build-up that can greatly reduce morbidity and complication rates.

Ultrasound (US), with frequencies above 20 kHz, can activate the auditory system through a cochlear mechanism that can be caused from fluid vibrations and transduction within the cerebrospinal fluid (CSF). Thus, varying the US stimulation parameters and activation conditions can cause CSF vibrations with varying cochlear and auditory activation. The present disclosure leverages this new US-based technique to vibrate the CSF and/or cochlear fluids, which provides another dimension of perturbation of the auditory system. The recorded OAEs, which are dependent upon changes in ICPs, will depend on whether or not ultrasound is applied, as well as the variable ultrasound patterns, center frequencies, and amplitudes. Thus, novel apparatuses and diagnostic methods will be described herein, which provide noninvasive ways to assess hearing function and intracranial pressure (ICP). The described methods may be especially useful for noninvasive assessment of hearing and ICP in newborns and provide noninvasive methods for identifying intracranial hypertension in adults and children, which could expedite the diagnosis of brain and spinal pressure build-up and greatly reduce morbidity and complication rates. The disclosed methods also provide new research tools to study the hearing system and brain or CSF pressure effects.

Hearing function is usually measured by presenting auditory sounds to the ear and measuring various outcome signals. This includes physiological signals or neural signals, for which electrodes are placed on the head or ear region to measure the sound-induced activity (e.g., auditory brainstem responses, (ABR), middle latency responses (MLR), cochlear responses/electrocochleography, and other EEG responses). The physiological or neural signals can indicate the function of the hearing system, such as hearing thresholds and suprathreshold function. The nonlinear properties of the cochlear function (e.g., activity of inner and outer hair cells) can be characterized by measuring sounds by microphones in the ear in response to sound stimulation of the ear. The active and nonlinear properties of the cochlear system cause sounds to be generated back out of the ear because the sounds coming into the ear activate the cochlea in an active/feedback-controlled way. This includes what is known as otoacoustic emissions (OAEs), which specifically relies on feedback/active signals from the outer hair cells within the cochlea to finetune how the cochlea sensitively and accurately processes incoming sound signals.

### Fundamentals of Human Hearing

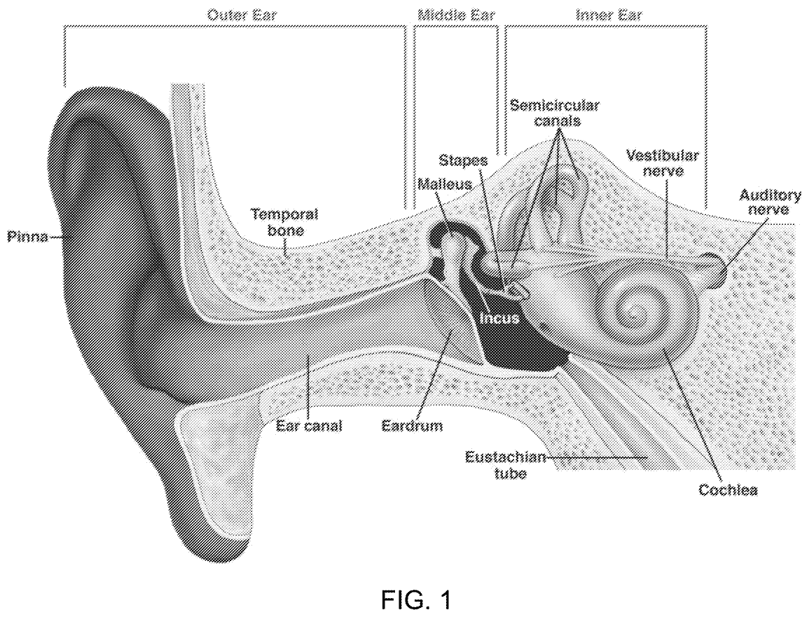

Hearing involves the perception of sound waves in a structure called the cochlea before being passed to the brain, specifically the auditory cortex. Sound waves must be converted to electrical signals before reaching the brain either through air or bone conduction. With air conduction, the pathway starts with sound waves entering the outer ear. The sound waves contact the tympanic membrane causing mechanical vibrations that are passed to the communicating ossicles (malleus, incus, and stapes) and lead to amplification of the initial sound wave. The ossicles contact the round window of the cochlea causing the fluid inside the cochlea, composed of endolymph and perilymph, to vibrate (FIG. 1). Along one portion of the cochlea, called the basilar membrane, the vibrating endolymph causes voltage-gated channels within hair cells to open thereby leading to depolarization and increased neuronal firing rate. The hair cell depolarization leads to activation of auditory nerve fibers that pass through various nuclei in the ascending auditory pathway before reaching the auditory cortex where sound can be perceived. In contrast to air conduction, bone conduction mainly includes vibrations that are passed from the temporal bone to the cochlea therefore bypassing the tympanic membrane. The perception of different tones, or sound wave frequencies, involves unique organization of the cochlea and neuronal structures.

Humans are normally able to perceive sounds between 20 Hz and 20,000 Hz. The cochlea has tonotopic organization, thus, sound waves with higher frequencies activate hair cells at the base of the cochlea whereas low frequency sounds stimulate the apex. The underlying mechanism of tonotopic organization in the cochlea involves differential traits in the basilar membrane. At the base, the basilar membrane is tuned for high frequencies whereas the apex is tuned for low frequency sounds that are more readily sensed. Tonotopic organization also occurs with the ascending auditory pathway structures such as auditory nerve, brainstem, midbrain, and auditory cortex.

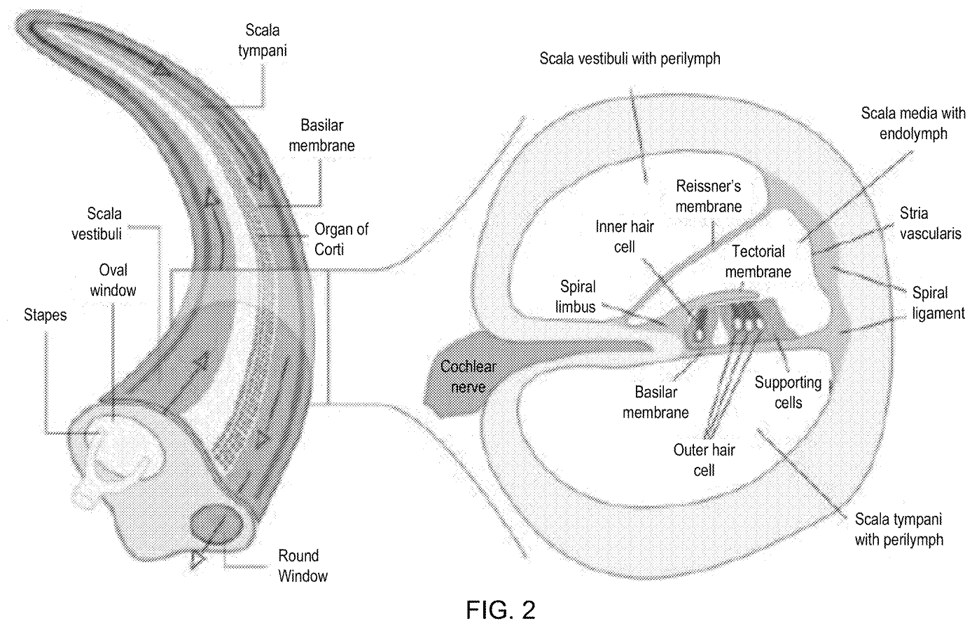

There are two populations of hair cells within the cochlea-inner and outer hair cells (FIG. 2). Inner hair cells (IHCs) are the sensory receptors and responsible for converting the incoming fluid wave to a neural signal in the auditory nerve by depolarizing. Outer hair cells (OHCs) work by contracting and elongating in response to efferent signals to amplify the fluid wave thus increasing the sound sensitivity and frequency resolution of the cochlea. The OHCs underline the cochlear amplifier which is a non-linear process due to their active movements. Otoacoustic Emissions as a Hearing Measure

There is a plethora of methods for assessing objective hearing status. The strategies can be divided into active and passive processes. Active methods rely on responses from the subject to indicate hearing level such as pure-tone audiometry, which involves presenting sound at multiple frequencies and determining when the subject can perceive the sound or word recognition tasks. Alternatively, passive methods do not require patient interaction and include auditory brainstem responses (ABRs), electrocochleography, and otoacoustic emissions (OAEs). Importantly, OAEs are unique in that they are non-invasive. Hence, OAEs are commonly part of the newborn hearing exam administered after birth to detect congenital hearing loss.

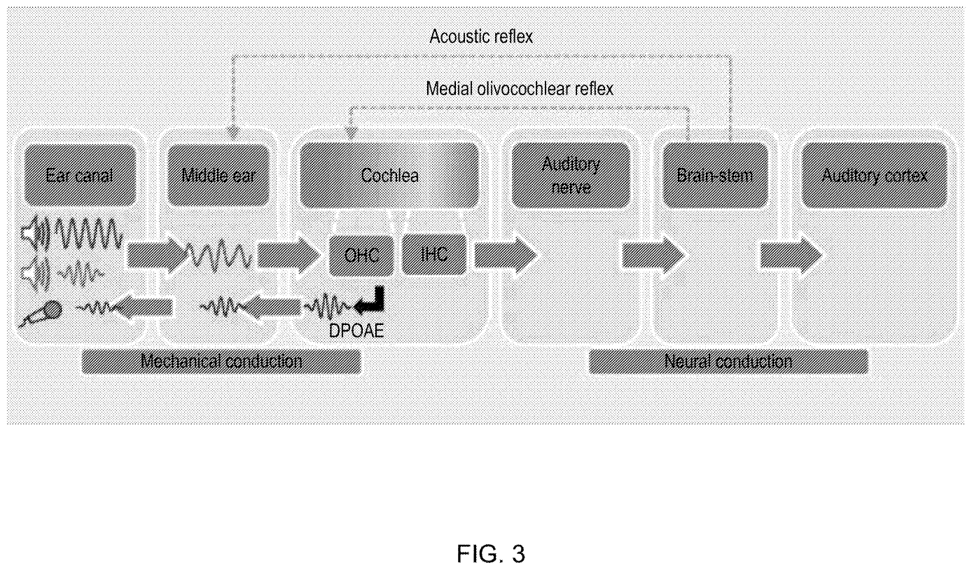

OAEs arise from the outer hair cells of the cochlea and were first described in 1978. As previously stated, OHCs can amplify incoming sounds at the cochlea through their active movements. The OHC movements, as part of the cochlear amplifier, and interaction with the basilar membrane generate a low-level fluid wave within the cochlea that travels retrograde through the middle ear and to the outer ear (FIG. 3). Thus, OAEs can be detected with a microphone placed in the outer ear. In sensorineural hearing loss, outer and inner hair cells are damaged leading to both impaired amplification and sensation of sound.

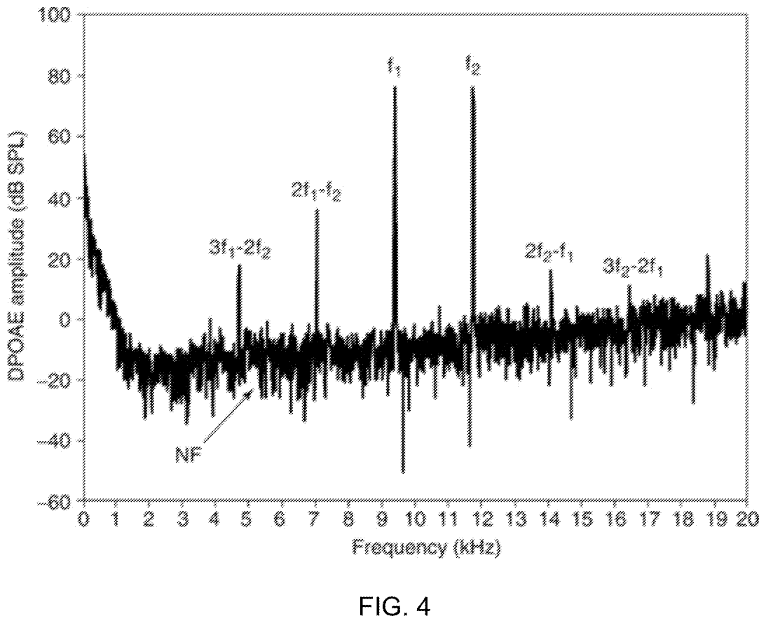

The two primary methods of eliciting OAEs are evoking with an acoustic stimulus or monitoring for spontaneous production. Spontaneous otoacoustic emissions (SOAE) are continuously produced by the cochlea without an acoustic stimulus with frequencies between 500 and 4,500 Hz. Conversely, Distortion Production Otoacoustic Emissions (DPOAEs) use simultaneous presentation of two pure tones (f₁ and f₂). The pure tones activate the cochlear amplifier, and outer and outer hair cell non-linearity leads to an audible signal that can be sensed by a microphone placed in the outer ear canal. For the optimum DPOAE response, the ratio f₁/f₂ should preferably be set to 1.2 with a primary frequency (f₁) between 1000 and 8000 Hz. However varying f₁/f₂ ratios can also be used and effective, and frequencies can be up to 10000 or higher depending on device capabilities for humans. In animal research the frequencies can be above 30 or 40 kHz depending on the species. Since the cochlear amplifier is non-linear, the resultant DPOAE response has a frequency consistent with an intermodulation product such as 2f₁−f₂ (cubic distortion product), 3f₁−2f₂, and 4f₁−3f₂ (FIG. 4). DPOAEs provide an objective measure of outer hair cell functionality that correlates with hearing ability. In current practice, DPOAE responses are elicited by acoustic stimulation via air conduction, for example, to assess newborn hearing.

### Ultrasound Stimulation and Cochlear Activation

Until recently, it was assumed the auditory system responded to a band of acoustic frequencies denoting the audible range (200 Hz to 20,000 Hz). However, ultrasound (US) stimulation, defined by frequencies greater than 20,000 Hz, can activate the auditory pathway up to the auditory cortex via a cochlear pathway. For example, 220 kHz transcranial US stimulation leads to both cortical and subcortical activation. Mechanistically, when cochlear fluids were removed or the auditory nerve transected in experiments, US was not able to activate the same regions. Hence, it was demonstrated that US leads to cochlear activation via an intracranial cerebrospinal fluid (CSF) pathway. Some components of the pathway may include a bone conduction pathway that then vibrates the fluids in the cochlea. This activation changes the mechanical properties of the middle ear to cochlea transfer functions. Additional studies have demonstrated that removal of CSF diminishes responses during acoustic bone conduction which gives additional support to a CSF pathway in US stimulation. Notably, neither study described cochlear hair cells responding to frequencies outside the audible range, rather, there may be a nonlinear transformation of US stimulation that produce audible frequencies and ultimately involve cochlear hair cells as well.

Interestingly, these various sound-induced measurements can be affected by pressure build-up in the fluids of the brain, which are continuous with the fluids of the cochlea. Thus, this pressure build-up can change the vibration dynamics of the cochlea. Researchers have attempted to measure these changes in pressure effects in the cochlea and how they affect OAEs, DPOAEs, and other responses to sound in the audible range, which could potentially provide an indirect and noninvasive measure of intracranial pressure (ICP). The ability to measure ICP noninvasively would be a critical and clinically important solution since currently, measuring ICP requires invasive surgery to open up the skull and directly measure pressure in the brain or spinal cord using various pressure measuring apparatuses. This can be especially challenging or risky for children who have pressure build up in their head or spinal area, and surgical approaches are risky for young children and infants, as well as adults.

### Diagnostic Avenues of Ultrasound-Based Hearing Markers

Evidence of US stimulation producing indirect activation via an intracranial and cochlear pathway raises further questions on how the mechanism may be exploited for clinical benefit. US stimulation that can elicit diagnostic auditory responses like acoustic sound such as aforementioned OAEs and ABRs can provide novel targets for hearing loss evaluation. Such responses may be referred to as acoustic responses or auditory responses. In various implementations described by the present disclosure, such auditory responses (e.g., OAEs, ABRs, DPOAEs) can be evoked using acoustic stimulation, ultrasound stimulation, or a combination of both.

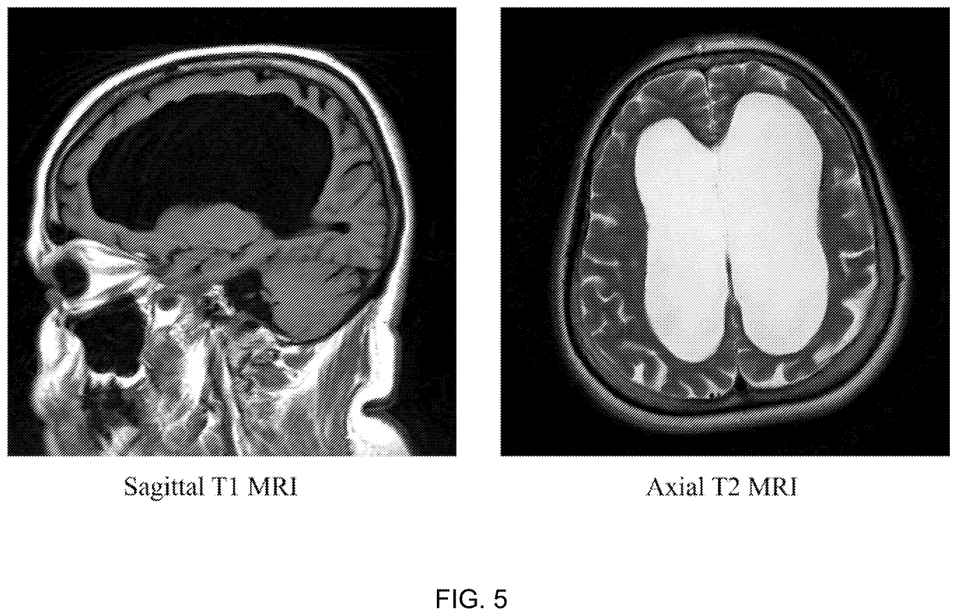

US stimulation does not activate the cochlea in the same way as sound stimulation through the outer and middle ears. Instead, US activates different parts of the cochlea with varying temporal patterns since it travels through a different mechanism or pathway that would not be possible or similar to how sound activates the cochlea. Thus, US stimulation can identify portions of the cochlea that are damaged or less sensitive to US stimulation, which would not be sensed by sound stimulation. Furthermore, the ability to identify the function of outer hair cells versus inner cells, as well as feedback reflexes or circuits (e.g., brainstem and midbrain to the cochlea, auditory nerves, and middle ear reflexes) is made possible with US stimulation. Such assessment would differ using sound stimulation because US stimulation activates the different subsets of hair cells in different ways compared to sound stimulation. Additionally, conditions that implicate intracranial pressure including hydrocephalus and intracranial hypertension, which may be caused by trauma, infection, congenital abnormalities, or metabolic abnormalities, may correlate with US-based hearing measures. These various sound-induced measurements can be affected by pressure build-up in the fluids of the brain and thus could have a mechanism for diagnosis (FIG. 5).

Currently, the clinical standard for diagnosis of increased ICP involves magnetic resonance imaging and lumbar puncture with continuous monitoring by a ventricular or intraparenchymal pressure transducer with surgical placement. However, transducer placements are invasive with neurosurgery required while lumbar punctures cannot provide continuous measurement and often lead to complications such as neuropathy. Thus, a non-invasive continuous method of monitoring ICP, not unlike the concept behind pulse oximetry, would provide clinical benefit. The present disclosure describes the use of US stimulation to evoke an auditory response, which can indicate hearing function and be correlated with ICP. The system and method will be demonstrated with amplitude-modulated US that successfully causes DPOAEs in guinea pigs that are correlated to ICP. For example, data has shown that US can perturb the CSF, eliciting OAEs that change inversely with elevated ICP.

ICP measurement can be greatly improved by perturbing the CSF pressure with US, which directly affects the ICP. Using US, the pressure can be perturbed in a wide variety of ways, and the auditory (e.g., OAEs, DPOAEs, stapedius reflex, startle response, audiograms, etc.) and neural (e.g., electrocochleography, ABRs, MLRs, late latency responses, multi-channel EEG, etc.) responses can be measured. In contrast with previous methods, which provided a single set of transfer functions for a given ICP, the methods and systems described herein make it possible to perturb the ICP to different levels and oscillations around the current pressure value. This creates a wide range of transfer functions using OAE, or other auditory activation, such as middle ear responses and tympanometry, and neural recording methods. The variety of measured response data can be correlated with ICP values and other conditions to create a predictive model, such as a lookup table or machine learning algorithm, that is more accurate than current methods and can be used to noninvasively predict the ICP value for a given person or condition. The approach will become more accurate as more data are measured from a greater number of subjects and conditions to create a large database of values for the prediction model.

The DPOAEs or neural evoked responses can also be measured in response to US stimulation, with or without acoustic stimulation. The US energy applied to the head will activate the cochlea in a different or complementary way to that of the acoustic stimulation. Therefore, the use of US stimulation provides an orthogonal or overlapping set of transfer functions and responses for a given ICP value or condition. Thus, this multimodal approach provides a richer set of data to predict the ICP value. Furthermore, this multimodal approach can provide alternative information about the hearing health or function for a given individual that is not possible with acoustic stimulation. This opens up a novel set of diagnostic methods that uses US alone, US compared to acoustic stimulation, or US in combination with acoustic stimulation, not only for assessing hearing function but also to noninvasively measure ICP.

US stimulation may be applied to the head or body to vibrate the CSF. The US vibrations can be measured in another location of the head or body. For example, pulsations in the eyeball region or tympanic membrane can be measured using optical techniques that image the vibrations in the eye or ear drum. This can be done independently or in conjunction with OAE response measurements. When the ICP is high, there will be greater resistance to the vibrations in the CSF or body fluids, which alters the transfer function of the US stimulation response across the head or body. This alteration in resistance/impedance and transfer functions provide a surrogate for the changes in ICP. Through large data collection across many individuals and conditions, the modeling from these data can be used to predict the ICP values for individuals based on the observed transfer functions for varying US stimulation parameters.

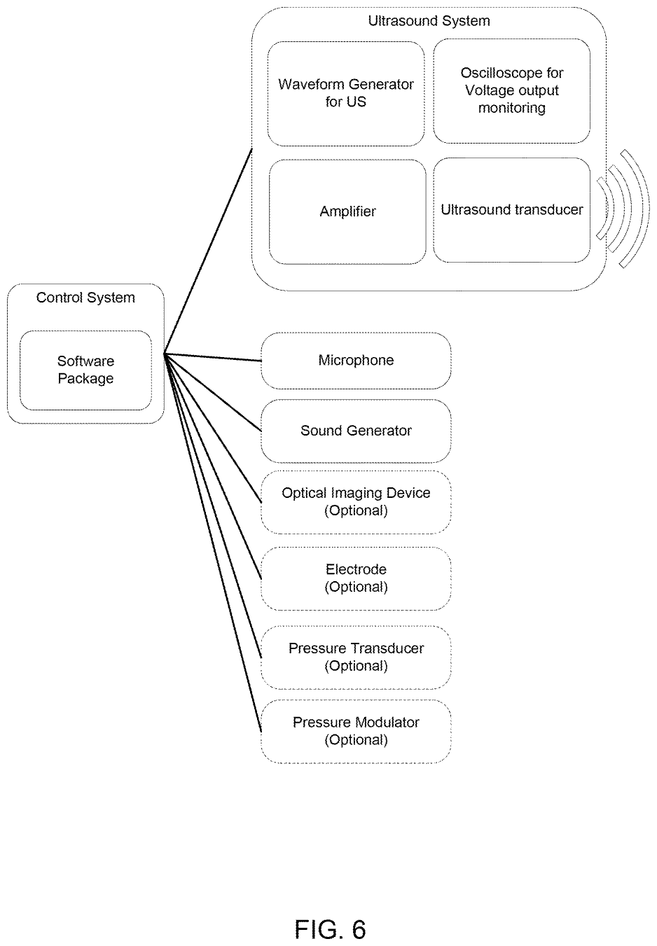

Referring now to FIG. 6, a system is illustrated for an embodiment of the disclosed system and methods for noninvasive multimodal auditory stimulation. The system may include a control system that may include one or more controllers. The control system may be used to define a pattern for ultrasound waves that can be defined by the user through a software package or defined otherwise by the control system. The control system can be in communication with and control one or more ultrasound transducers, an ultrasound system, one or more sound generators, and one or more microphones. The ultrasound transducers may be configured to be placed on or near the head or ear of a subject and generate ultrasound waves. The ultrasound waves may be frequency-modulated, amplitude-modulated, or both, with a wide range of parameters and modulation patterns available. The US signal can compromise of a carrier frequency in the range of 50 kHz to 5 MHz with a narrower range of 100 kHz to 1 MHz. This carrier signal can then be amplitude modulated with frequencies of 100 Hz to 20 kHz and one or two or more frequencies can be presented. More complex waveforms are also possible. Amplitudes can vary from 10 kPa to 2 MPa depending on the frequencies applied but a narrower range of 50 kPa to 500 kPa, 100 to 500 kPa, or 20 kPa to 100 kPa can be used as well. These signals should typically be ramped on and off to minimize onset/offset distortions. These stimuli can be repeated multiple times and durations can be 0.001 seconds to 1000 seconds each to allow sufficient averaging or estimation time for the output signals, but narrower range could be 0.005 to 5 seconds. The ultrasound system may include one or more oscilloscopes, which may be used for voltage output monitoring. The system can further include one or more amplifiers, such as dual amplifiers with 100 and 200 W power. The ultrasound system can further include and one or more waveform generators, which may each be coupled to an amplifier. As a non-limiting example, the ultrasound wave may have a frequency of 220 Hz with an intensity of 70 kPa. As another non-limiting example, the ultrasound waves may be delivered for 1 second durations over 5 repetitions with a delay of 20 seconds between presentations. The ultrasound waves may be produced in bursts of 1-10 ms duration repeated every 100 ms or using another duty cycle. As another non-limiting example, ultrasound may be produced for bursts of 1 s for any number of repetitions (e.g., 5 repetitions) with an acoustic level between 10- and 100-dB SPL, or more narrowly, 40- to 70-dB SPL.

The sound generator may be an external sound generator, such as a speaker. The sound generator may also be coupled with an ear insert, which can be placed inside a subject's ear. The sound generator may generate any sound pattern as an acoustic stimulus with various constant or changing frequencies and amplitudes. The sound pattern may be defined by the user via a software package of the control system, or otherwise determined by the control system. The control system may also determine and communicate the trigger of the sound generator. For example, the sound generator may generate acoustic stimuli that elicit DPOAEs, other OAE response, or any auditory response. The speakers may produce sounds anywhere in the audible range (e.g., 20-20,000 Hz). For example, to elicit DPOAEs, the stimuli may include two pure tones with the primary (f₁) frequency set at 5, 10, or 15 kHz with equal levels between 40- and 60-dB sound pressure level (SPL). The secondary frequency (f₂) may be set such that the ratio f₁/f₂ is within the range of 0.5 to 2, preferably where the ratio f₁/f₂ should is 1.2. The two acoustic stimuli may be presented for 5 to 10 repetitions of 1 second duration. The acoustic stimuli may include any frequency in the audible range of 20 Hz to 20,000 Hz at intensities of 0-150 dB. The acoustic stimuli can be presented for any number of repetitions, for example 1 to 1000 repetitions, with duration that may be in the range of 1 ms to 10 s. The acoustic stimuli may also be presented with frequency or amplitude modulation.

The microphone may be any suitable device which can record small audio signals with high enough sensitivity. It may be an external microphone or a small microphone that can be coupled with an ear insert or otherwise situated inside one or both ears of the subject. The microphone can be configured to record DPOAE, OAE, other auditory responses, or any other audio signal. The control system can control the trigger to acquire an audio signal, or the microphone can continuously acquire audio signals. The control system can also record the acquired audio signal, connect it to a timestamp, and store it for later analysis.

The control system may also be in communication with an optional optical imaging device. The optical imaging device may be a camera or other suitable device that can acquire images of one or both eyeballs or one or both eardrums of a subject. For example, the optical imaging device can record pulsations in the eyeball region or tympanic membrane by imaging the vibrations in the eye or eardrum. The optical imaging can be used to track the blood vessel vibrations or pulsations of the fluids in the structure, which can then be used to assess amount of vibration. For example, greater brain fluid pressure may lead to smaller vibrations. This data may contribute to the validation or calibration of the system. It may also be used to further characterize the DPOAEs and OAEs with or without ultrasound stimulation effects. The optical imaging device may record images at a frame rate faster than the physiologic vibrations, and may be configured to record continuously or triggered by the control system.

The system may also include one or more optional electrodes. The electrodes may be configured to be placed on the head or scalp of a subject to record a neural signal. For example, the electrodes may be used to measure sound-induced activity, such as auditory brainstem responses (ABR), middle latency responses (MLR), cochlear responses/electrocochleography (EEG), or other EEG responses. The electrodes may be in communication with the control system to record, localize, and analyze the EEG signals. These neural recordings can indicate the level of activation in the cochlea, and thus the auditory brain regions. For example, if the brain fluid pressure is high, the pressure may also be higher in the cochlea, which can cause the cochlear vibration to be reduced and lead to reduced brain activity. These effects can be used to validate or calibrate the system. The neural signals may also be used for further characterization of hearing function and brain fluid pressure in addition to OAEs and DPOAEs acquired with or without ultrasound stimulation.

The system may also include an optional pressure transducer for the purpose of validation or calibration of the system. The pressure transducer may be configured to measure intracranial pressure or a pressure of the fluid in the auditory system. The pressure transducer may, as a non-limiting example, have a sampling rate of 1 to 1000 Hz with 21-bit resolution and transmit the measured pressure to the software package or to be otherwise recorded by the control system. The pressure transducer should be configured to measure pressures within the range of 0 mmHg to 100 mmHg. Also, for calibration or validation, the system may include a pressure modulator that is configured to modulate ICP of a subject. Examples of systems and methods that may be suitable for ICP modulation will be described in further detail below. As non-limiting example, the pressure modulator may be configured to alter ICP from 1 to 50 mmHg, or within a narrower range of 5 to 25 mmHg. In some implementations, such ICP modulation can be performed in animal or phantom experiments for model calibration. Such ICP modulation may also be used to test US or acoustic parameters that produce significant ICP-dependent changes in OAEs, which can be used or modified in block 702, for example.

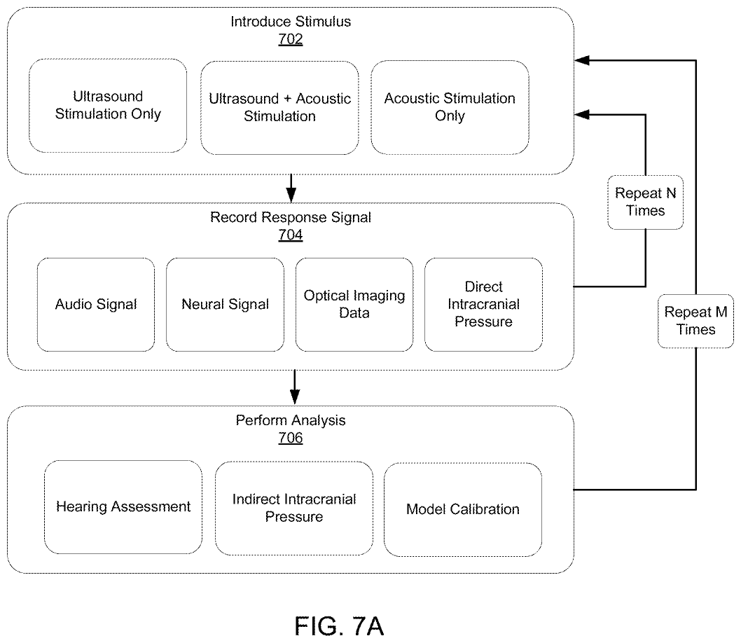

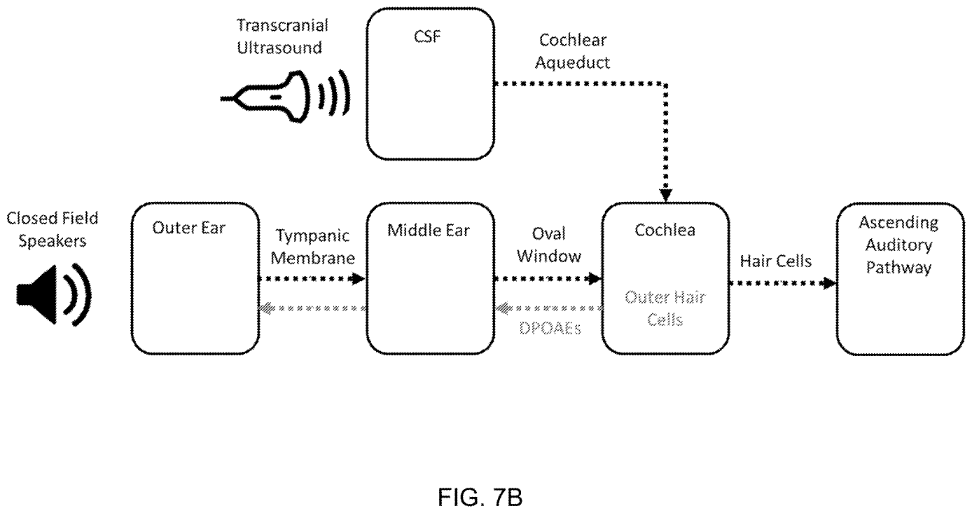

Referring now to FIG. 7A, methods for some embodiments are illustrated. The process may be carried out by the system shown of FIG. 6, for example. The process may include process blocks for introducing a stimulus, recording a response signal, and performing analysis. For example, introducing a stimulus may include introducing an acoustic stimulus only using a sound generator, introducing an ultrasound stimulus only using the ultrasound system, or simultaneously or sequentially introducing both the ultrasound and acoustic stimuli. Such acoustic or ultrasound signals can include pure tone sinusoidal waves of a single frequency or predominantly a single frequency. However, other wave patterns may be used. For example, multiple pure tones can be used simultaneously or in succession. In some configurations, the acoustic or ultrasound signals can be amplitude modulated, frequency modulated, or some combination thereof. More Complex patterns can also be presented, such as music-like signals or speech-like signals. In some configurations, the acoustic stimulus can be applied externally to the tympanic membrane. The control system may be used to coordinate and record the timing of the acoustic and ultrasound stimuli.

The physiological responses to the stimuli may be recorded in process block 704. For example, the recorded responses may include audio signals, which can be recorded by the microphone. For example, the audio signals may include DPOAEs, other OAE responses, or other auditory responses from the ear. The recorded responses may also include neural signals, which can be recorded by the electrodes to indicate localized neural activity or optical images, which can be acquired by the optical imaging device.

Intracranial pressure may also be directly measured by the optional pressure transducer in order to validate or calibrate subsequent analysis. For example, intracranial pressure can be directly measured in animal models or phantom experiments using a pressure transducer. In other implementations the intracranial pressure can be measured in human subjects or patients using clinical methods, such as lumbar puncture or intraventricular catheters.

The measurement of the recorded responses may be coordinated with the introduction of the stimuli by the control system or a user. Alternatively, the measured responses may be timestamped upon measurement. In this way, the control system can continually store the response with their specified times before, during, and after the introduction of the stimuli.

The introduction of the stimuli along with recording of the response may be repeated any number of N times with various stimuli or modifying any parameter of each stimulus (e.g., amplitude, frequency, duration, etc.).

The control system may use any combination of the audio signal, neural signal, optical imaging data, or direct intracranial pressure measurement to perform analysis in process block 706. The analysis of block 706 may include model or system calibration, model or system validation, hearing assessment of subjects, or invasive or noninvasive estimation of intracranial pressure.

As a non-limiting example, the direct intracranial pressure measurement may be used with the audio and US induced responses to perform model calibration. The direct intracranial pressure measure can be measured over time, which may have different values at different times. The sound and US-induced measures described above can be also measured over time, in which their specific outcomes can be correlated or calibrated to the ground truth values of the direct pressure measurements, leading to a model or calibration function. Once the model is generated, other sound and US-induced outcomes can be interpolated and predicted from the model or calibration function. Additional data, including demographic and characteristics data, from groups of patients will further refine a generalized model or calibration function across many patients. As one non-limiting example, the audio signals produced within the ear (e.g., OAE, DPOAE) can be calibrated to the direct intracranial pressure measurements using a machine learning algorithm, such as a neural network, by forming a lookup table, or by another mathematical model (e.g., regression). In some implementations, audio signals can be measured for a group of subjects or patients receiving invasive ICP measurement (e.g., via surgery, lumbar spine puncture, or intraventricular catheter) providing audio signals paired to ICP measurements. This paired data can be used as training data for model calibration.

The model calibration can additionally take optical imaging data and neural signals into account. The audio signals can be used to perform a hearing assessment of the subject, which may be supplemented by neural signals or optical imaging data. Some combination of the audio signal, neural signal, or optical imaging data may also be used to indirectly measure intracranial pressure using a model obtained from calibration or accessed via the control system. As a non-limiting example, artificial intelligence may be used to analyze audio signals (e.g., DPOAE response), neural signals, optical imaging data, or some combination thereof to assess hearing loss or intracranial pressure. Additionally, such data can be used to evaluate damage to the cochlea, identify less sensitive regions of the cochlea, assess function of various subsets of hair cells (e.g., inner ear vs. outer ear), or assess feedback reflexes or circuits. In this way, a machine learning algorithm may be trained on various combinations of US-evoked auditory responses, acoustic-evoked auditory responses, bimodal-evoked auditory responses, optical imaging data, and neural signals. Training such models may include measuring ground truth data (e.g., invasive measures of hearing assessment, direct intracranial pressure, subjective hearing assessment, animal model data). Training machine learning models may also include supervised or self-supervised learning.

Thus, the process is capable of noninvasively assessing hearing or intracranial pressure for a given subject. The process can also be used to collect calibration data to train or otherwise inform a model for future noninvasive hearing assessment or estimation of ICP. For example, the model may be trained to relate any combination of noninvasive data (e.g., audio signal, neural signal, optical imaging data) to an invasive, direct measure of intracranial pressure. As a non-limiting example, the model could include a machine learning algorithm, such as a neural network (e.g., convolutional neural network, recurrent neural network, generative adversarial network, multi-layer perceptron, or similar). The network can be trained on a wide variety of directly measured intracranial pressure or hearing assessment measures and audio signals over a wide range of stimuli parameters. The model may also be trained using neural signals, optical imaging data, or both. The model may also provide a lookup table to estimate ICP by comparing the audio signal measured for a given subject with ranges set by the audio signals measured for other subjects. The model may also be determined by regression or other known methods. For example, the ICPs can be numerically fit and analyzed to produce a transfer function that can predict the ICP for a given ultrasound input and OAE measurement.

By collecting and characterizing the relationship between the audio signals, neural signals, or optical signals measured in response to stimuli over various parameters with intracranial pressure measured directly from more comprehensive and invasive methods (e.g., via a pressure transducer), it will be possible to predict absolute or clinically relevant values of ICP noninvasively for future subjects. The flexibility of the wide range of available stimuli parameters (e.g., amplitude, frequency, pattern, repetition rate, duration, etc.) of acoustic stimulation, ultrasound stimulation, or both can provide an accurate model over a wide range of conditions. Similarly, the relationship between the audio signals, neural signals, optical signals, or combinations thereof with various measures of hearing health can be used to characterize hearing health in new subjects. Harnessing ultrasound stimulation of the auditory pathway can provide a wide range of measurement parameters (stimulation patterns, amplitude, frequency, and so forth) and stimulation combinations (acoustic, ultrasound, acoustic+ultrasound, or a combination thereof) that allows for a more complete assessment of hearing function than use of acoustic stimulation alone.

After forming the model, lookup table, etc. the control system can access the model or lookup table in process block 706 to characterize hearing ability, other hearing metrics (e.g., hair cell function, cochlear damage) or intracranial pressure for a subject based on the signals measured in process block 704 and the stimuli produced in process block 702. In this way, the auditory response can be compared to a database that includes previously measured auditory responses that correspond with directly measured hearing metrics or intracranial pressure values obtained for a group of subjects. In other implementations, the auditory response can be analyzed based on the model that was trained on paired intracranial pressure data and auditory response data in order to non-invasively estimate intracranial pressure. Similarly, the auditory response data can be analyzed based on a model that was trained on paired hearing function data (e.g., cochlear damage, hair cell function, hearing sensitivity, hearing frequency range, audiogram) and auditory response data in order to non-invasively and objectively estimate various measures of hearing function.

The analysis in process block 706 may also include quality control. If the measured signal is not sufficient for clinical or research analysis, the process may be repeated any number of M times. Further, the analysis in block 706 may inform the parameter sets for further tests required and repeat the process (e.g., M times) with stimuli parameters that are controlled based on the initial analysis.

Referring now to FIG. 7B, an example system is shown as it may be used clinically or in research interacting with a physiological system. Such setup may be used for hearing exams (e.g., newborn hearing exam) or for measuring ICP. Ultrasound can activate the cochlea through a cerebrospinal fluid (CSF) pathway. This pathway may include cochlear aqueducts, as shown in FIG. 7B. Additionally, the pathway may include vibrations through vestibular aqueducts, vibration of fluids through the auditory nerve pathway into the cochlea, or vibration of fluids in porous bone. In this way, the activation pathway may include vibration of CSF that vibrates cochlear fluids, such as through cochlear aqueducts, vestibular aqueducts, the auditory nerve region of the cochlea, and so forth. Amplitude-modulated (AM) ultrasound can induce frequency-specific activation, eliciting DPOAEs, which are produced by the outer hair cells in response to pure-tones. DPOAEs arise due to the non-linearity of the cochlear amplifier.

### EXAMPLES

### Example 1

### Methods

### Acoustic and Ultrasound-Evoked Otoacoustic Emissions in Guinea Pigs

Guinea pigs (GPs) were chosen as the animal model for their larger size than mice or rats and use in previous US stimulation studies within the laboratory. During all experiments, GPs were under general anesthesia with alternating doses of pure ketamine and ketamine/xylazine in a 4:1 mixture. Every 15 minutes, heart rate, pulse oximetry, and core body temperature were recorded. Reflexes, including toe-pinch and blink, were elicited every 15 minutes in addition to vital signs thus indicating the need for intra-operative anesthesia. All animal protocols and surgical procedures were approved by the Institutional Animal Care and Use Committee (IACUC).

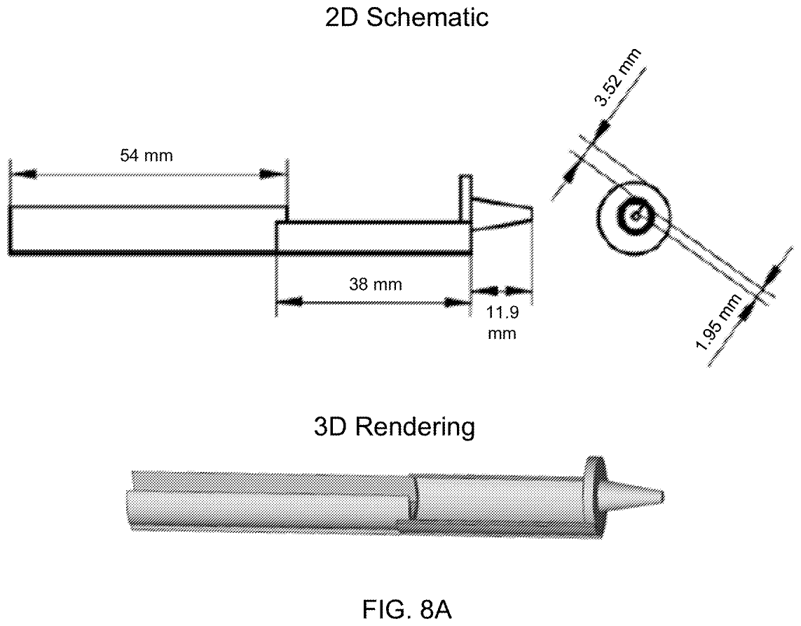

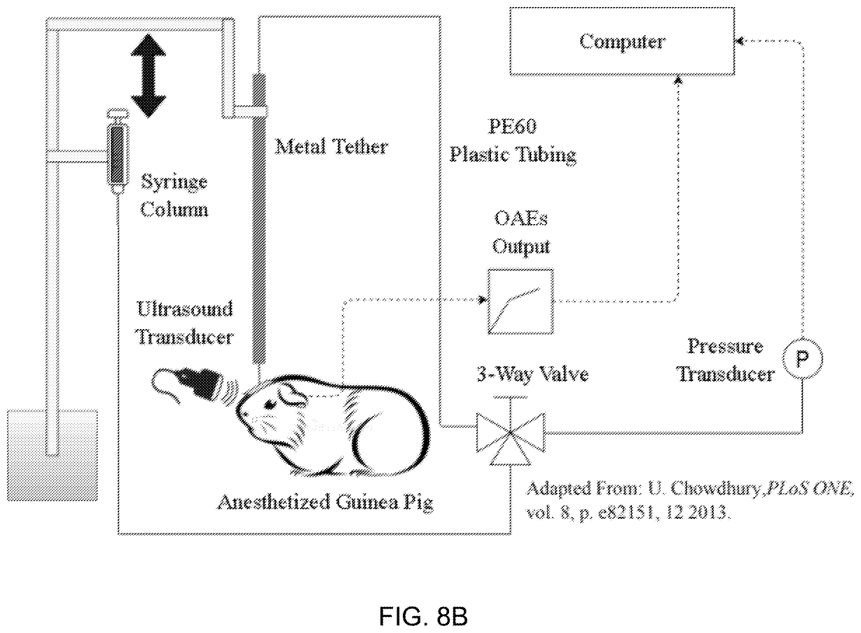

First, a setup was developed that could consistently and effectively produce acoustic DPOAEs. The equipment included two closed-field speakers, stereotaxic apparatus or frame including one each 10 cm metal and 3D printed ear-bar and a bite bar, and a probe microphone. The probe microphone was coupled to the speakers. The system was controlled by a computer system (e.g., a MATLAB-capable computer with Synapse software). Early experiments attempted to present acoustic stimuli at the opening of the 10 cm metal ear-bar but resulted in excessive interference and poor sensitivity to the low-level DPOAEs due to the path length. Hence, custom 3D printed ear-bars were created that were formed to the probe microphone as well as the guinea pig outer ear enabling the microphone to be situated close to the cochlea. FIG. 8A shows a non-limiting example of an ear-bar that may be used in accordance with the present disclosure. The ear-bar may be 3D printed or otherwise molded or formed.

After the GP was anesthetized and cerumen cleared out of the outer ear, the GP was placed in the stereotaxic apparatus with both ear-bars inserted. To elicit DPOAEs, two pure tones were presented with the primary (f₁) frequency set at 5, 10, or 15 kHz with equal levels between 40- and 60-dB SPL. The two acoustic stimuli were presented for 5 to 10 repetitions of 1 second duration. The responses recorded by the microphone were averaged over all repetitions before applying a Fournier transform to determine the frequency response demonstrating the presence or absence of a DPOAE.

### Stability and Input/Output Characteristics

The application of US-evoked DPOAEs to diagnosis of hearing loss or intracranial conditions requires stability of the DPAOE signal. Following from signal theory, the DPOAE system should ideally be time-invariant. That is, assuming a normal hearing subject, presentation of identical acoustic or US stimuli at any given time must lead to similar DPOAE responses. Otherwise, the strategy would be less reliable at providing diagnostic information. The time-invariant traits of the system were tested by recording DPOAEs for primary frequencies 5, 10, and 15 kHz for both acoustic and US-evoked DPOAEs over a period of 15 minutes in a normal hearing GP. Acoustic stimuli were presented at 60 dB while US was set at 70 kPa. Stimuli were delivered for 1 second duration for 5 repetitions with a delay of 20 seconds between presentations.

Modulation of Intracranial Pressure with Correlating Otoacoustic Emissions

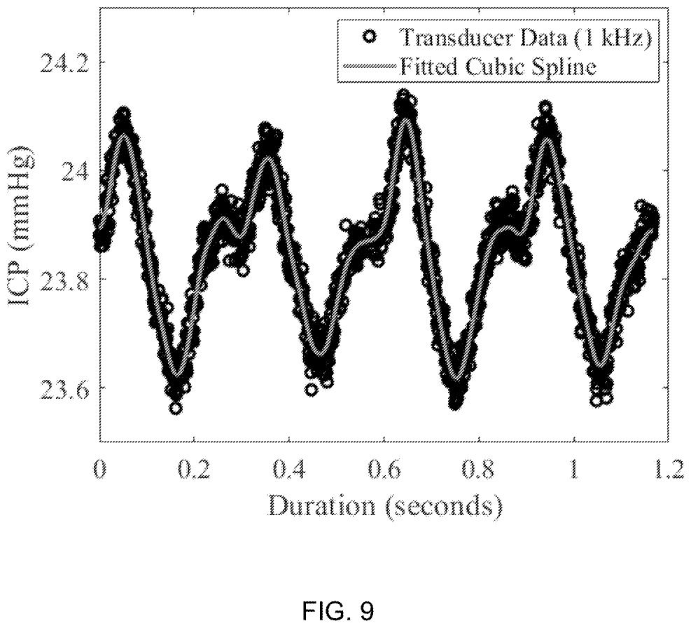

There are multiple methods of modulating ICP experimentally in animal models. Previous studies have utilized chemical-induction with kaolin; however, the implementation was not conducive to finely controlling or rapidly normalizing ICP. Thus, we develop a hydrostatic approach that was preferred in literature experiments that desire sensitive ICP control and continuous monitoring. The method involved connecting a suspended syringe column, plastic tubing, and pressure transducer via a three-way valve and filled with saline or artificial CSF (FIG. 8B). A metal or plastic tether was attached to the exposed GP cranium overlying a craniotomy and sealed and adhered with epoxy resin. The pressure transducer has a sampling rate of 1 to 1000 Hz with 21-bit resolution that transmits to a software monitoring program. Confirmation of ICP monitoring can be completed by utilizing a high sampling rate and observing the ICP waveform (FIG. 9). ICP was modulated by raising or lowering the syringe column in relation to the GP tragus. With the pressure transducer placed at the level of the tragus, the recorded hydrostatic pressure equals the ICP.

In humans, normal ICP ranges from 5 to 15 mmHg. When ICP rises to above 25 mmHg, intracranial hypertension begins with risk of death from hypoxia at approximately 60 mmHg. However, there is a paucity of literature that equates similar reactions in GPs. We select three ICP levels, 5 mmHg (baseline), 15 mmHg (moderately elevated), and 25 mmHg (intracranial hypertension) for modulation in GPs.

### Results

### Characteristics of Multi-Modal DPOAEs

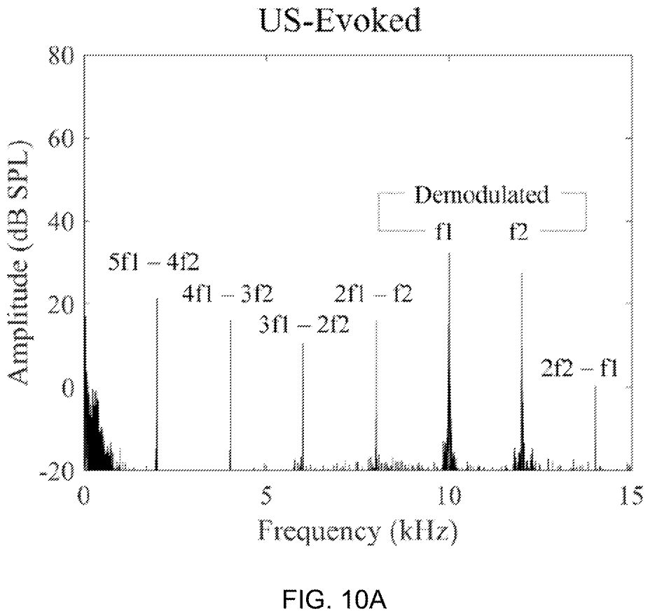

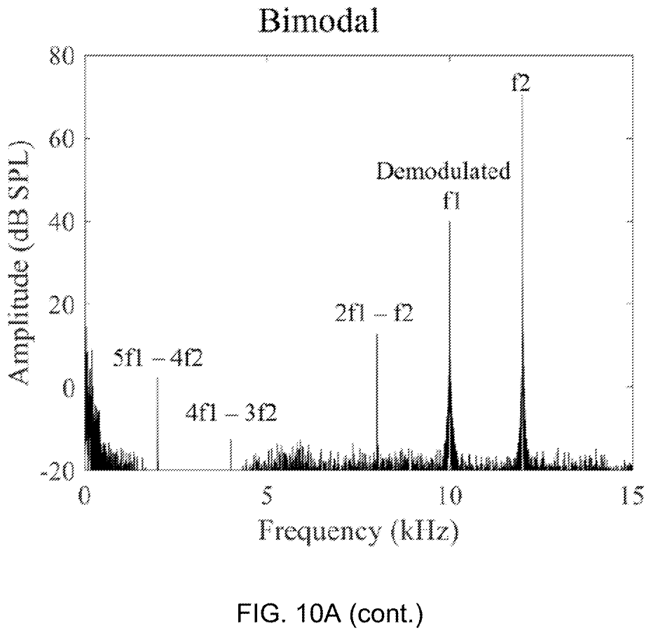

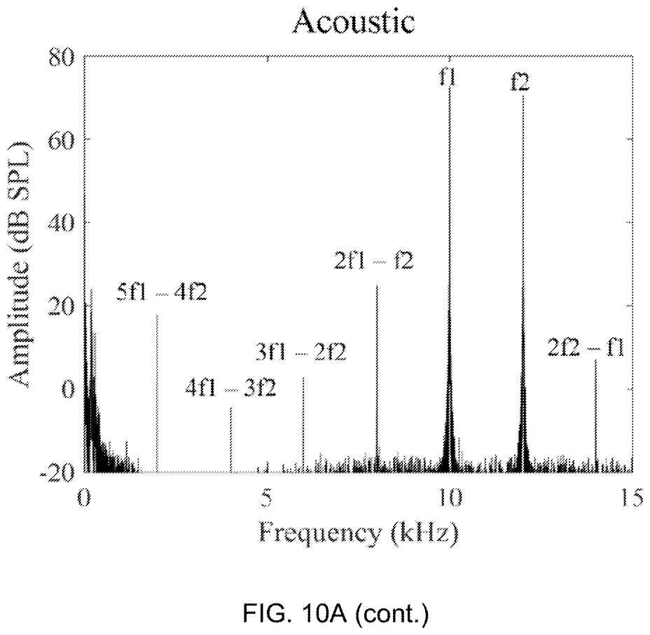

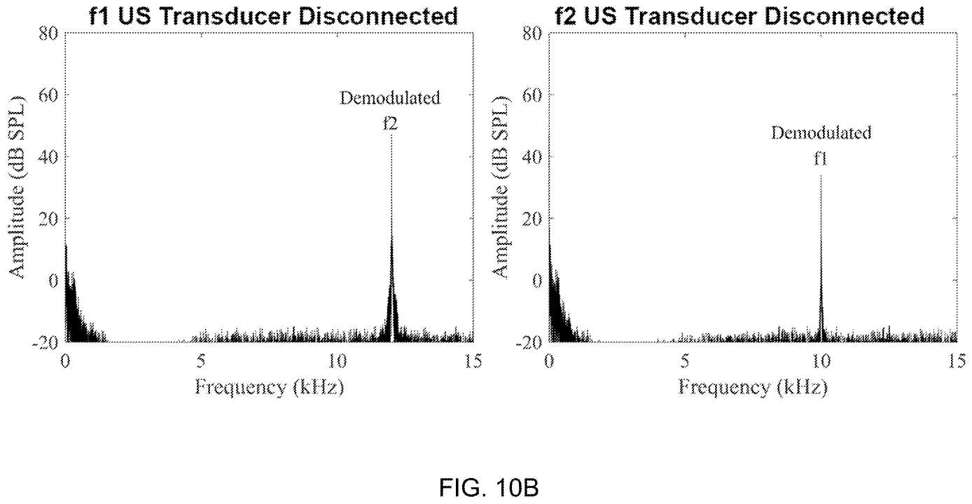

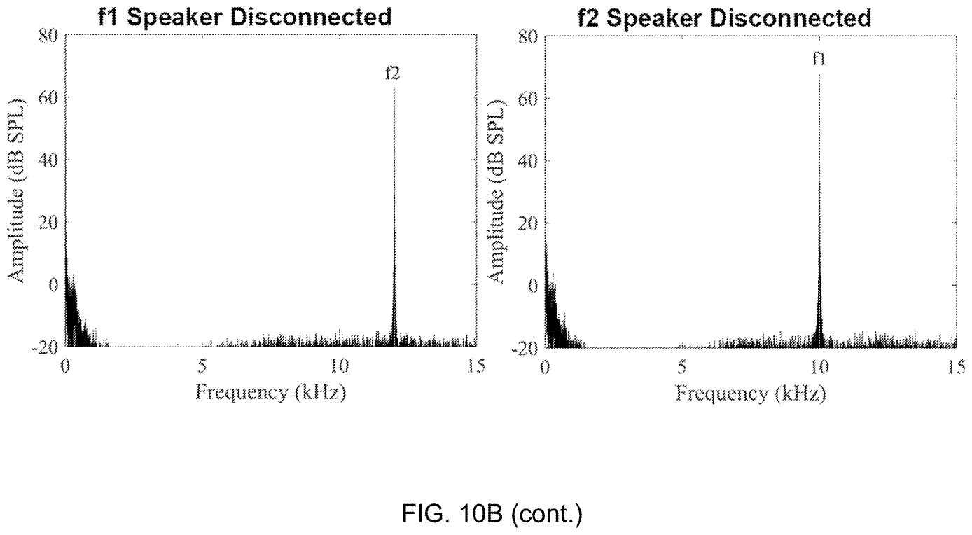

Initial results with acoustic DPOAEs demonstrated a frequency spectrum with peaks at expected frequencies including the prominent cubic distortion product 2f₁−f₂. Within the US-evoked and bimodal settings, the cubic distortion product was also produced as well additional intermodulation products thereby demonstrating the DPOAE response (FIG. 10A). Notably, the demodulated US component was shown in both spectra. With removal of one stimulus component, all spectra were void of intermodulation products which shows, as expected, that two pure-tone stimuli are needed to elicit a DPOAE.

The trial parameters associated with FIG. 10A are provided below:

| | | | | | Bimodal | |
|---|---|---|---|---|---|---|
| | | | | | (Acoustic and | |
| Parameter | Acoustic | | US-Evoked | | US stimulation) | |
| f1 frequency | 10 | kHz | N/A | | N/A | |
| f1 Modulated | N/A | | 10 | kHz | 10 | kHz |
| Frequency | | | | | | |
| f1 Carrier | N/A | | 220 | kHz | 220 | kHz |
| Frequency | | | | | | |
| f1 Level | 60 | dB SPL | 60 | kPa | 60 | kPa |
| f2 Frequency | 12 | kHz | N/A | | 12 | kHz |
| f2 Modulated | N/A | | 12 | kHz | N/A | |
| Frequency | | | | | | |
| F2 Carrier | N/A | | 100 | kHz | N/A | |
| Frequency | | | | | | |
| f2 Level | 60 | dB SPL | 60 | kPa | 60 | dB SPL |
| Duration | 1 | second | 1 | second | 1 | second |

| | | | |
|---|---|---|---|
| Repetitions | 5 | 5 | 5 |

FIG. 10B provides results from control experiments. In one experiment, shown on the top, the acoustic evoked DPOAE experiment was repeated after removing the acoustic speaker associated with f₁ (left) and again after removing the acoustic speaker associated with f₂ (right). Removal of either speaker eliminated the cubic distortion product DPOAE. In another experiment, shown on the bottom, the ultrasound evoked DPOAE experiment was repeated after removing the ultrasound transducer associated with f₁ (left) and again after removing the ultrasound transducer associated with f₂ (right). Removal of either ultrasound transducer eliminated the cubic distortion product DPOAE.

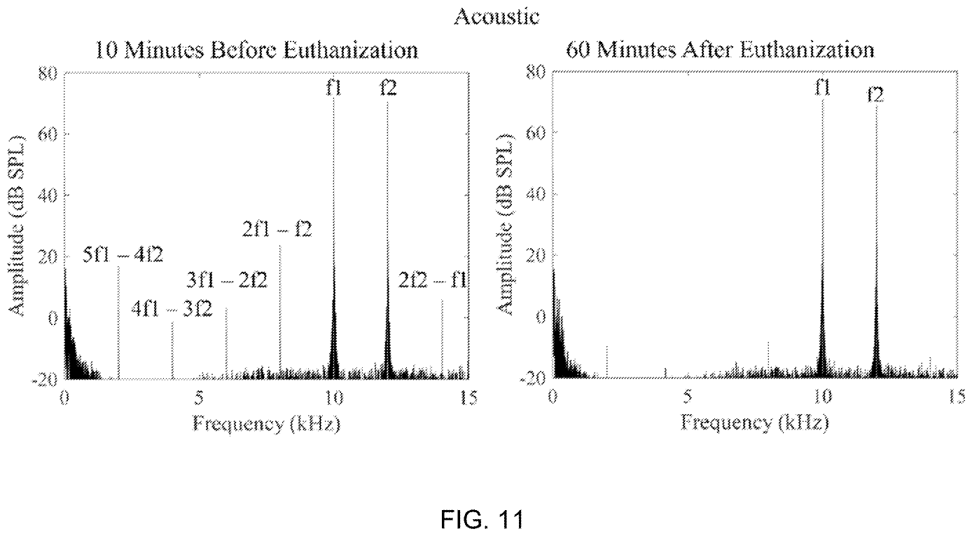

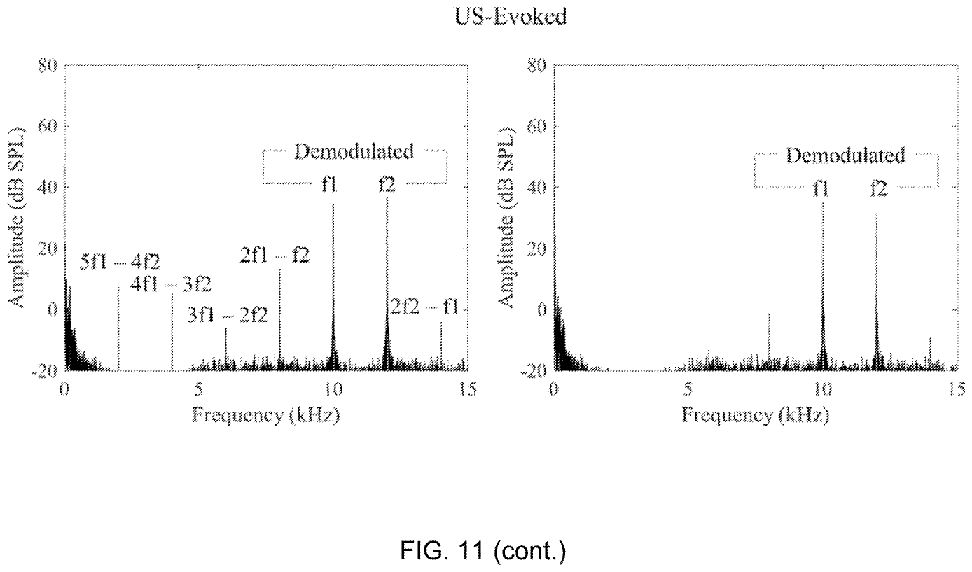

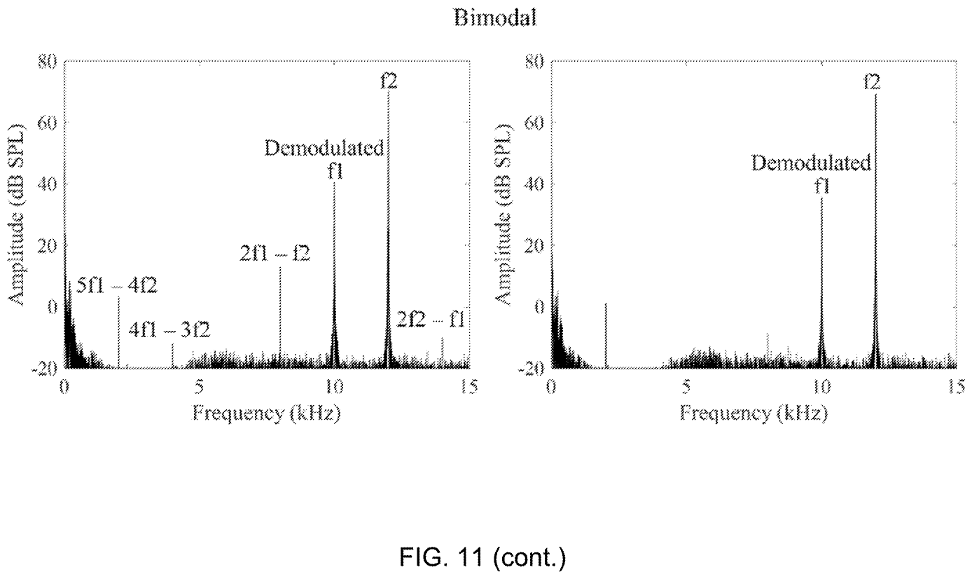

For further confirmation that DPOAEs are being elicited, as opposed to artifact intermodulation, frequency spectra were recorded before and after euthanization. To ensure that OHC death occurs after euthanization, 60 minutes are passed before recording the DPOAE response. As shown in FIG. 11, diffuse reduction in intermodulation peak amplitude, especially the cubic distortion product, occurred after euthanization. Nonetheless, there remains an artifact at select frequencies likely due to spurious intermodulation not attributable to OHC activation.

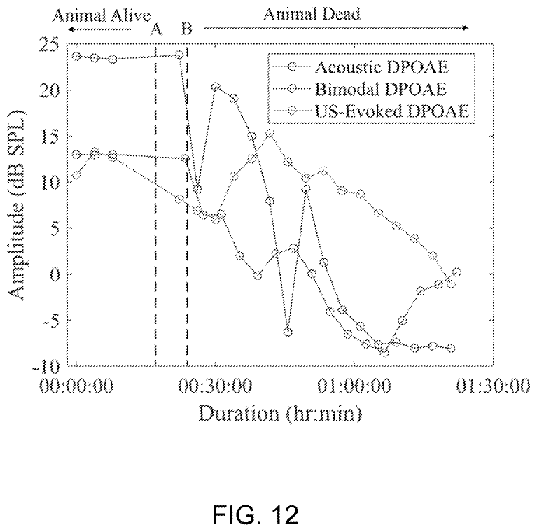

The decline in OHC activity after euthanization can be analyzed further by looking at the temporal trend of the DPOAE response. FIG. 12 demonstrates that all three modalities resulted in a down sloping DPOAE cubic distortion product amplitude consistent with progressive reduction in OHC functionality. The DPOAE amplitude range denotes the maximal reduction in OHC activity which for the acoustic, US-evoked, and bimodal modalities was −31.9, −16.4, and −21.5 dB SPL, respectively.

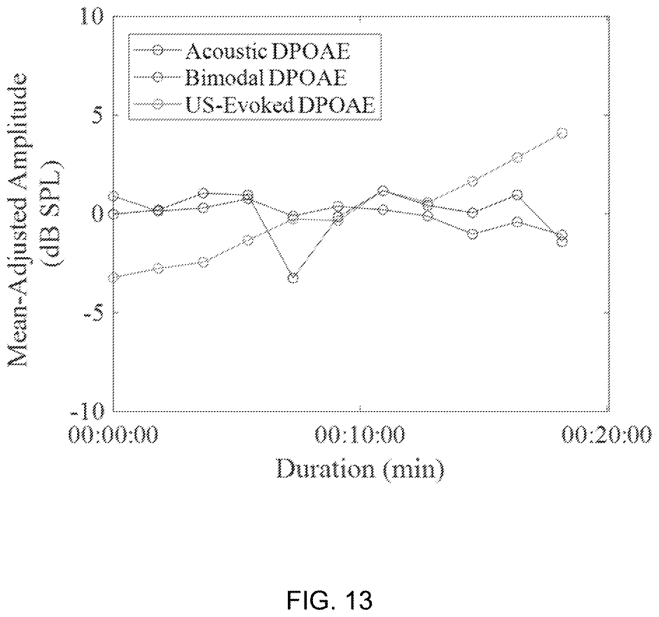

For DPOAE responses to be utilized in a diagnostic capacity, the signal time-invariancy is an important consideration. The time-invariancy reflects both the consistency of OHC responses as well as the robustness of the experimental setup. For DPOAE responses measured under constant conditions for 20 minutes, the standard deviation of the acoustic, US-evoked, and bimodal settings was 1.3, 0.64, and 2.35 dB SPL, respectively (FIG. 13).

### DPAOE Reponses as a Diagnostic Marker for ICP

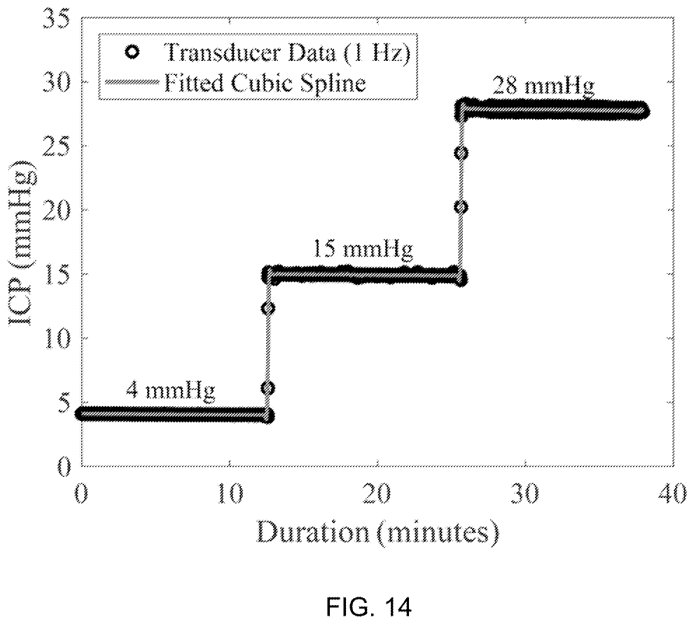

Three guinea pig experiments were initially completed for modulation of ICP. One experiment suffered from consistent leakage at the guinea pig cranial attachment while another resulted in an irreversible Cushing's reaction. Major changes were made for both the experimental ICP range and hydrostatic column adherent for the third trial which demonstrated stable ICP levels. As demonstrated in FIG. 14, ICP was increased step-wise at pressures of 4 (baseline), 15, and 28 mmHg. For a sampling rate of 1 Hz, the digital pressure transducer measurements indicated stable ICP at each step (FIG. 14).

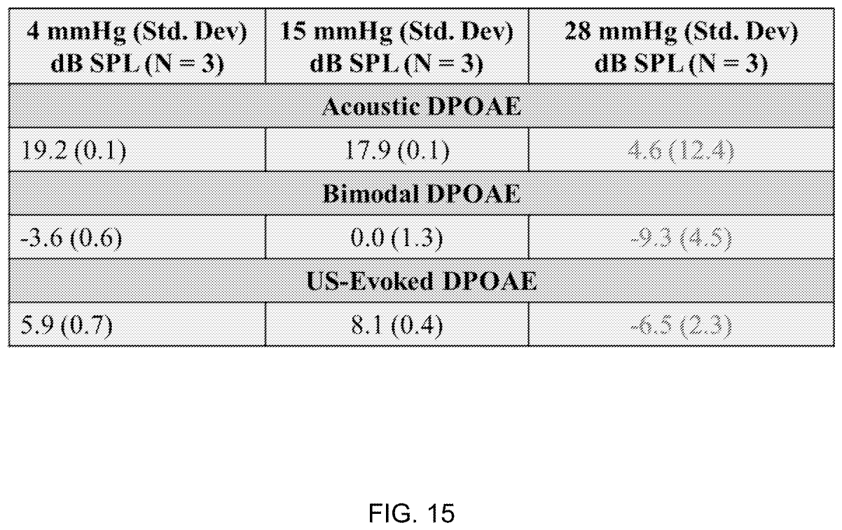

The DPOAE cubic distortion product response for each ICP step was recorded for all modalities. As shown in FIG. 14, moderate elevations in ICP (15 mmHg) demonstrated slight changes bidirectionally depending on the modality. However, at significantly elevated ICP (28 mmHg), there was a significant reduction in DPOAE amplitude for all modalities (FIG. 15).

### Discussion

DPOAEs have clinical relevance for the measurement of hearing ability due to being non-invasive, passive, and relatively quick to test. Hence, DPOAEs are important for the newborn hearing examination and determines if referral for secondary hearing evaluation may be necessary for congenital hearing impairment. However, DPOAEs have only been elicited with acoustic stimulation in previous literature. With US demonstrated to activate the cochlea and associated hair cells, OHC activation and therefore DPOAE generation was shown to be possible. Our research demonstrated that DPOAEs are produced in response to AM US with two different modulating frequencies and developed an experimental setup for time-invariant measurement.

US-evoked and bimodal DPOAEs have a variety of applications not possible with acoustic DPOAEs. US-based DPOAEs could be utilized as a hearing marker alongside acoustic DPOAEs or audiograms when evaluating auditory status. Traditional hearing evaluation methods perform poorly with detecting select instances of subjective hearing impairment known as “hidden” hearing loss. US-based DPOAEs may provide insight into these challenging hearing impairment cases. Furthermore, unlike acoustic stimulation which has a well-characterized limit for auditory damage both acutely and chronically, US activation of the cochlea does not. The correlation of US-evoked or bimodal with acoustic DPOAE amplitude could assist in developing initial guidelines for safe US stimulation levels.

Furthermore, US transduction to the cochlea was shown to occur with a fluid pathway via CSF. Leveraging fluid conduction could provide insight into conditions that implicate ICP such as hydrocephalus. Our results demonstrated that severely elevated ICP levels lead to drastically reduced DPOAE amplitude for all modalities. Previous research has demonstrated the reduction of acoustic DPOAE amplitude with elevated ICP, likely due to reductions in cochlear blood flow and altered middle ear transmission. The advantage of our approach is that DPOAE amplitude can vary continuously across different ICP elevations. Future experiments that utilize higher carrier frequencies also can have a more sensitive sensitivity to ICP changes.

### Conclusion

Multi-modal stimulation paradigms that extend beyond traditional acoustic presentation can activate the cochlea and produce OAEs. The elicitation of DPOAEs can provide direct insight into the pathway including the fluid conduction through CSF. Furthermore, US-based DPOAEs hold diagnostic potential for characterizing and assessing US auditory activation and conditions with elevated ICP.

### Example 2

### Background

Background: Distortion Product Otoacoustic Emissions (DPOAEs) are sounds generated by the outer hair cells of the cochlea in response to pure tones. Facilitated by the non-linear mechanism of the cochlear amplifier, DPOAEs reflect the integrity of the outer hair cells and thus are a robust and non-invasive method for hearing assessment, especially as part of the newborn hearing exam. Previously, DPOAEs have been studied with air-conduction in humans. Recent studies have demonstrated that transcranial ultrasound (US), particularly amplitude-modulated (AM) US, can stimulate the cochlea through a fluid pathway. We aimed to determine if AM US can also elicit DPOAEs thereby providing a novel diagnostic method for hearing assessment and measurement of intracranial pressure (ICP) that has been described previously for acoustic pure-tones.

### Methods

Five guinea pigs were utilized as an animal model for generating acoustic and AM US-based DPOAEs. Acoustic DPOAEs were recorded with primary (f1) frequency of 10 kHz at a level of 60 dB with two speakers each presenting a pure tone at a ratio of 1.2 to the secondary frequency (f₂) for 1-second duration. AM US-evoked DPOAEs were generated with two US transducers at carrier frequencies 100 kHz and 220 kHz coupled to the ipsilateral cranium approximately 1 cm rostral to the pinna with 70 kPa level. Modulated frequency of 10 kHz was applied to the carrier waveform for the primary frequency also at a ratio of 1.2. In both modalities with a single guinea pig, DPOAEs were recorded before and after euthanization. ICP was elevated hydrostatically with a column in communication with the subarachnoid space via a small craniotomy and DPOAEs were recorded at ICP steps of 4, 15, and 28 mmHg in a single guinea pig experiment.

### Results

Acoustic, US, and bimodal stimulation generated DPOAEs at the 10 kHz primary frequency at intermodulation frequencies of 8, 6, 4, and 2 kHz. The cubic distortion product (8 kHz) was most prominent with levels between 10- and 20-dB SPL. The gradual cessation of DPOAEs after euthanization confirmed that the signals are otoacoustic emissions with reductions of 16- and 32-dB SPL for acoustic and AM US stimulation, respectively. When ICP was elevated from 4 to 15 mmHg, DPOAEs in all modalities remained stable but further increases to 28 mmHg caused diffuse reductions in DPOAE amplitude with mean decrease of 13.3- and 14.6-dB SPL for acoustic and AM US-evoked modalities, respectively.

### Conclusions

We demonstrate that transcranial AM US generates DPOAEs that may be used as a hearing assessment method via a fluid pathway not utilized by conventional audiometry. With additional research to determine the optimum stimulation protocols, US-evoked DPOAEs may also be useful for noninvasive measurement of human ICP and diagnosis of intracranial hypertension.

### Example 3

### Introduction

Studies have shown that cochlear activation can be achieved via transcranial ultrasound stimulation. DPOAEs have been shown to reflect cochlear health and activation. Thus, DPOAEs can provide an output measure for determining ICP, which can be used to monitor intracranial hypertension. The systems and methods disclosed herein can be used to produce a model that connects input ultrasound stimulation to DPOAE output and ICP.

### Methods

Experimental setup for acoustic-evoked and ultrasound-evoked DPOAEs was developed using a guinea pig model. Such setup included sound processing from an external auditory canal microphone. The frequency ranges can be chosen based on the auditory range of the subject species (e.g., guinea pig, mouse, rat, human, and so forth). Experimental results showed that path length between the subject's ears and microphone and speaker can affect the signal intensity of the DPOAEs. Thus, the microphone and speaker may be placed in close proximity to the tympanic membrane. For example, 3D-printed ear bars can be used to allow the DPOAE microphone and speaker to be situated near the tympanic membrane, which can increase signal intensity and decrease artifact and noise. In some experiments, ear bars can be removed and the tympanic membrane can be directly coupled to the microphone or speaker using tubing. Such setup can reduce interference from standing waves that cause distortion, especially at higher stimulus durations.

DPOAEs were produced over a range of frequencies with f2/f1 set as 1.2. f1 was chosen between 8 kHz and 12 kHz with a duration of 1 s time-averaged over eight repetitions. The amplitude was set between 45-70 dB for both stimuli. The two tone stimulus duration was set as 2 ms, 5 ms, 200 ms. or 1 s. Longer stimulus durations can preferably enable better frequency resolution and reduce distortion.

The ICP was modulated using a hydrostatic pressure applied from an external column with an in-line digital pressure transducer. ICP was also directly measured using a pressure transducers connected to subdural space. The pressure transducers can be industrial or single-use medical grade. In one experimental example, a transducer with a sampling rate of 1000 Hz, resolution of 21 bit, and range of 0 to 2 bar was used. For some experimental setups, the pressure transducer used was an in-line digital pressure transducer (e.g., GD4200-USB available from ESI Technology Limited). Further setup details are available in FIG. 8B.

The ultrasound pressure level was determined based on a voltage between 0 to 100 mV and an amplifier of 100 or 200 W. The ultrasound stimulation was delivered as an amplitude-modulated waveform with a carrier and modulated frequency.

### Results

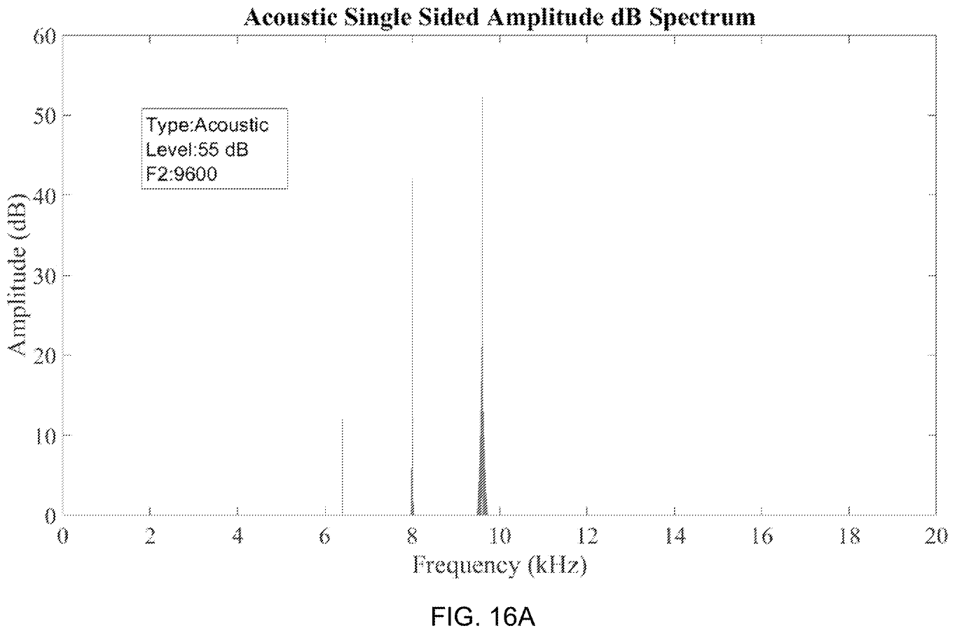

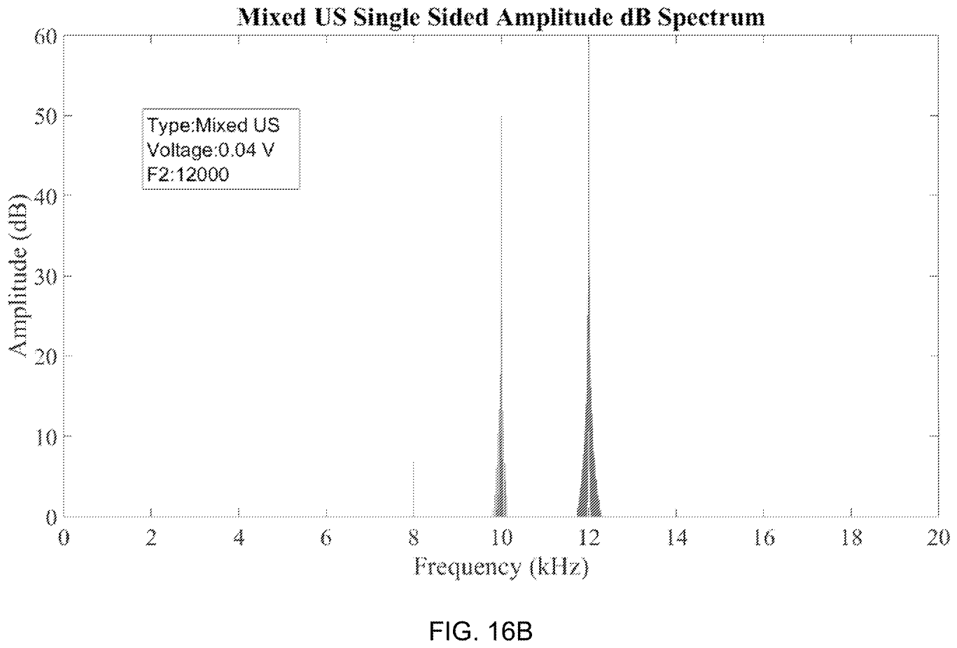

FIG. 16A shows an example acoustic-evoked DPOAE using frequencies of f1=8 kHz and f2=9.6 kHz with an intensity of 55 dB. FIG. 16B shows an example mixed or bimodal DPOAE using an amplitude modulated ultrasound with a carrier frequency of f1=10 kHz and an acoustic signal with a frequency of f2=12 kHz. The signal intensity was set to 0.04 V.

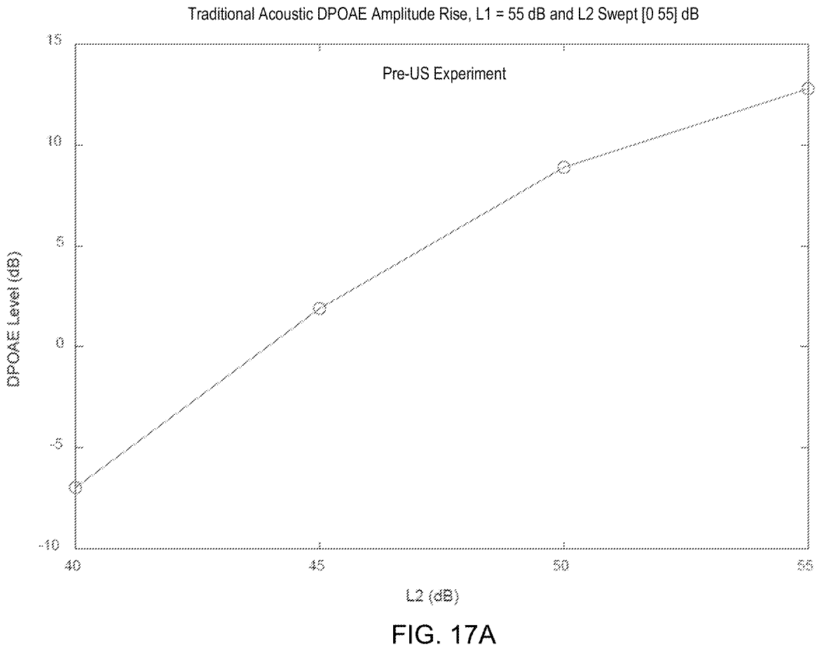

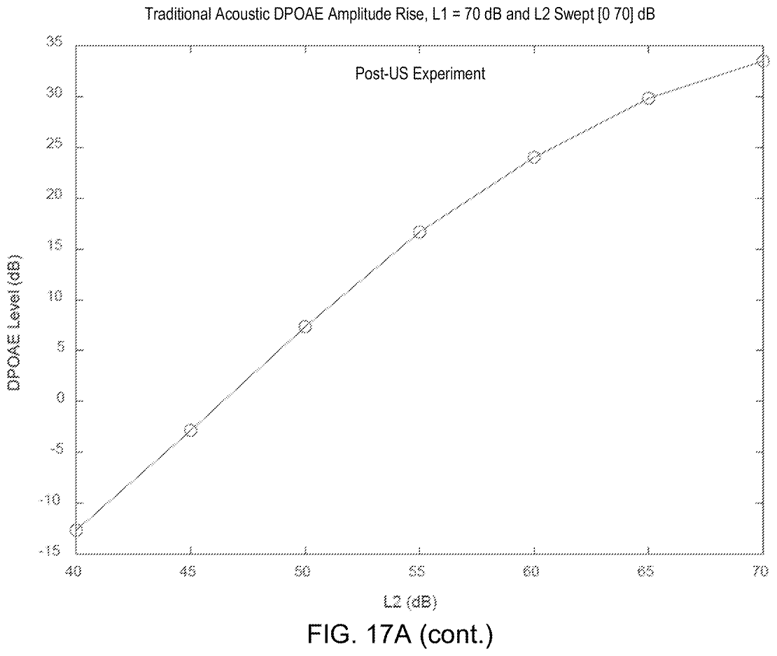

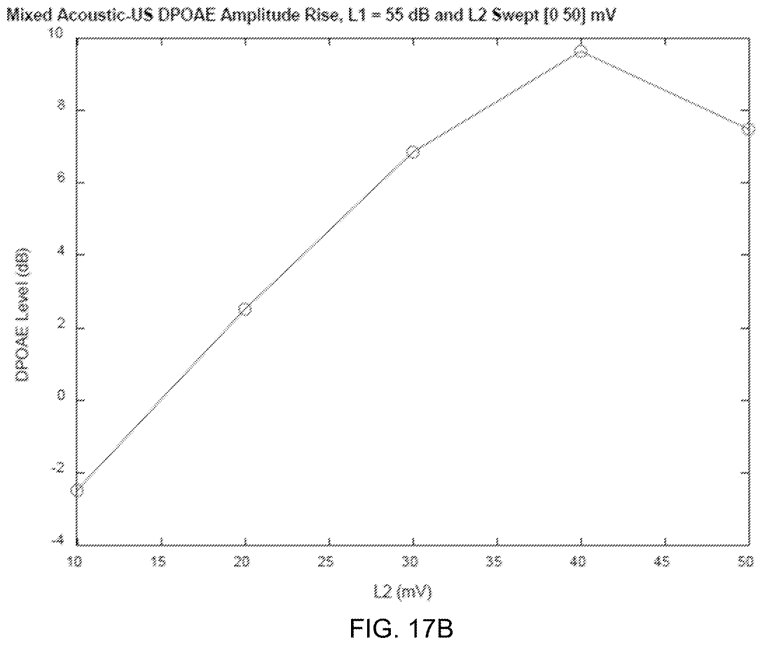

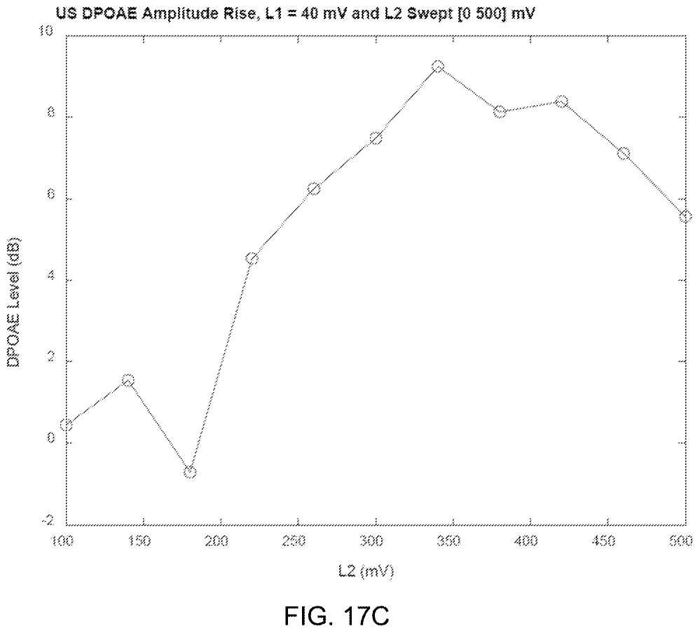

FIG. 17A shows that acoustic-evoked DPOAEs scale with the amplitude of either individual speaker used in the experimental setup. FIG. 17A also shows that the ultrasound stimulation used does not negatively impact hearing function, as the DPOAEs did not change before and after ultrasound stimulation. FIG. 17B shows that bimodal (e.g., acoustic and ultrasound) evoked DPOAE scale with ultrasound power input until reaching a nadir within a similar range as acoustic evoked DPOAEs. FIG. 17C shows that ultrasound evoked DPOAE scale with ultrasound power input until reaching a nadir within a similar range as acoustic evoked DPOAEs.

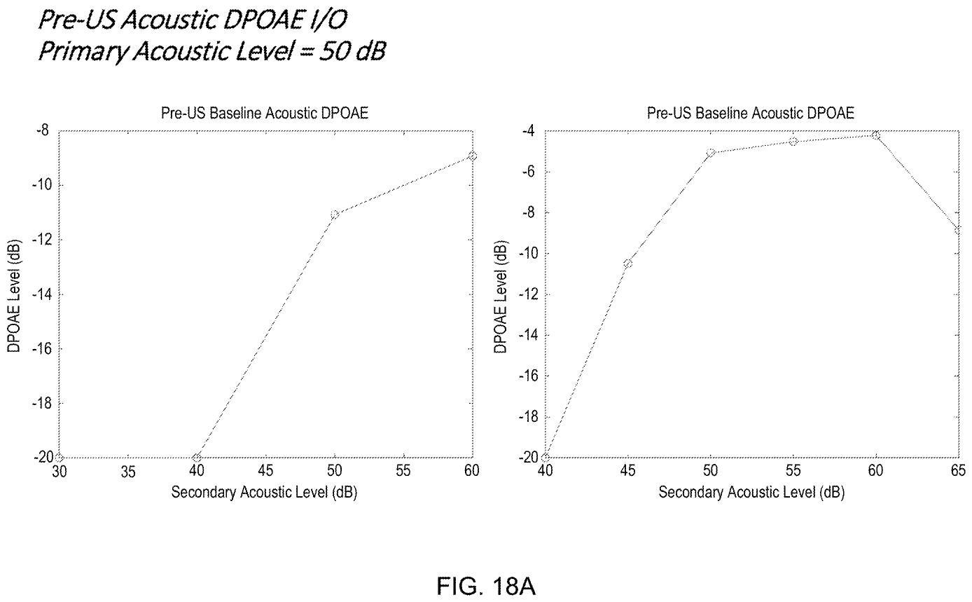

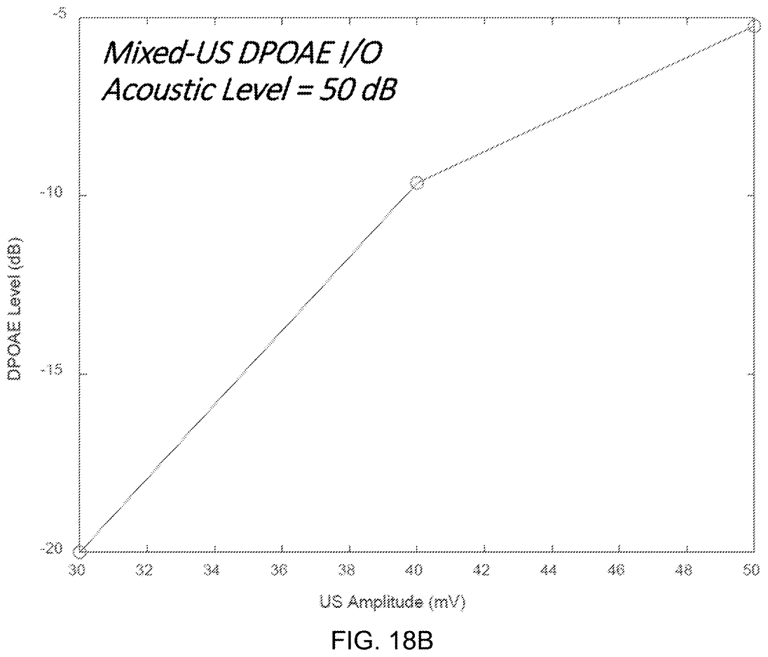

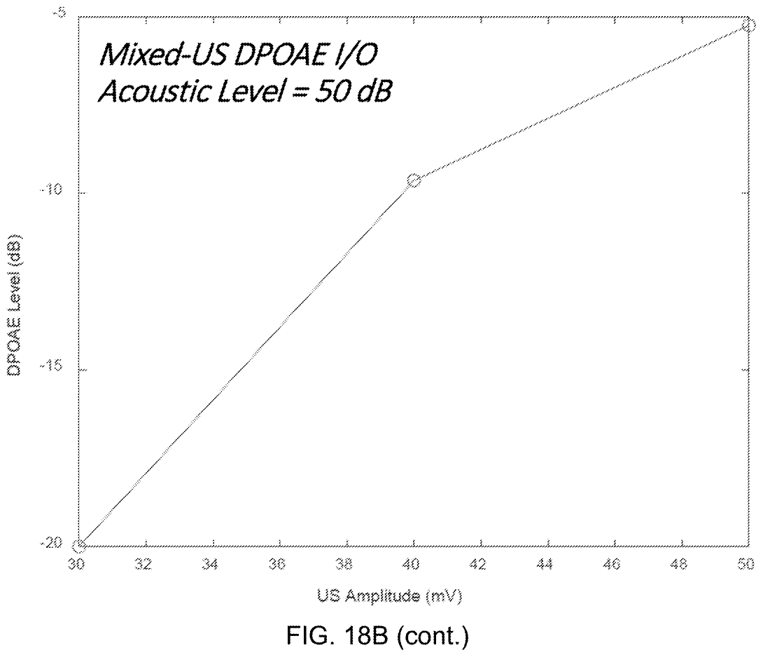

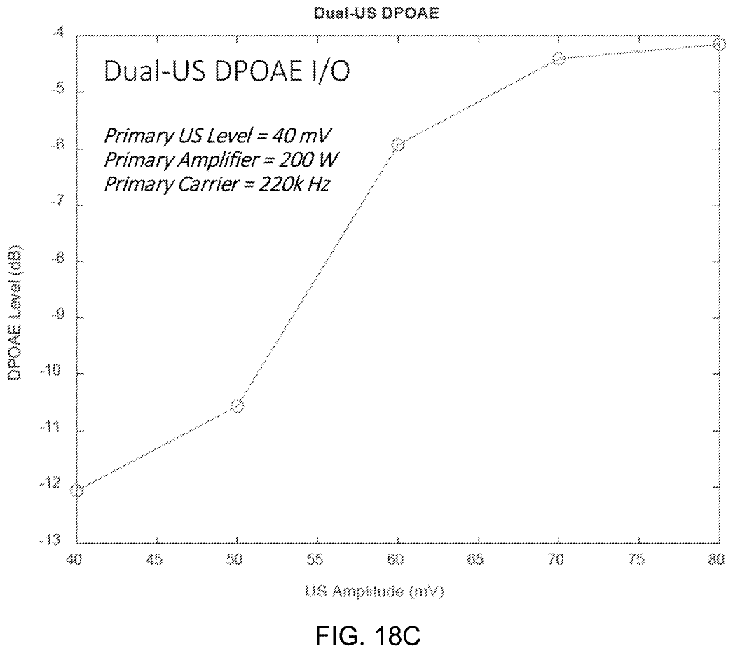

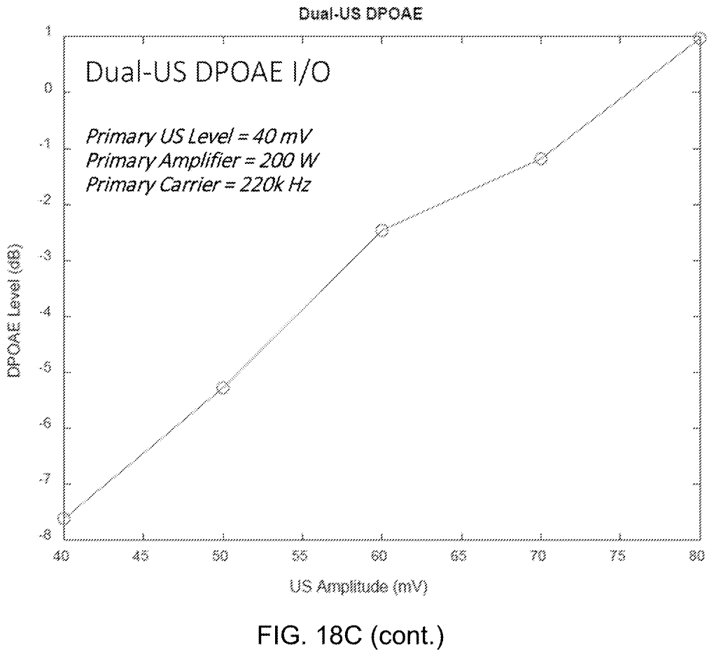

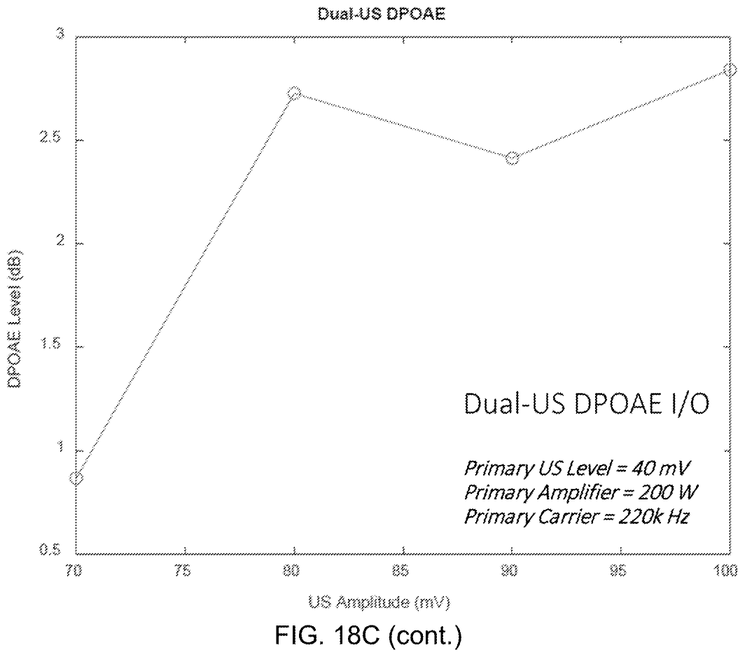

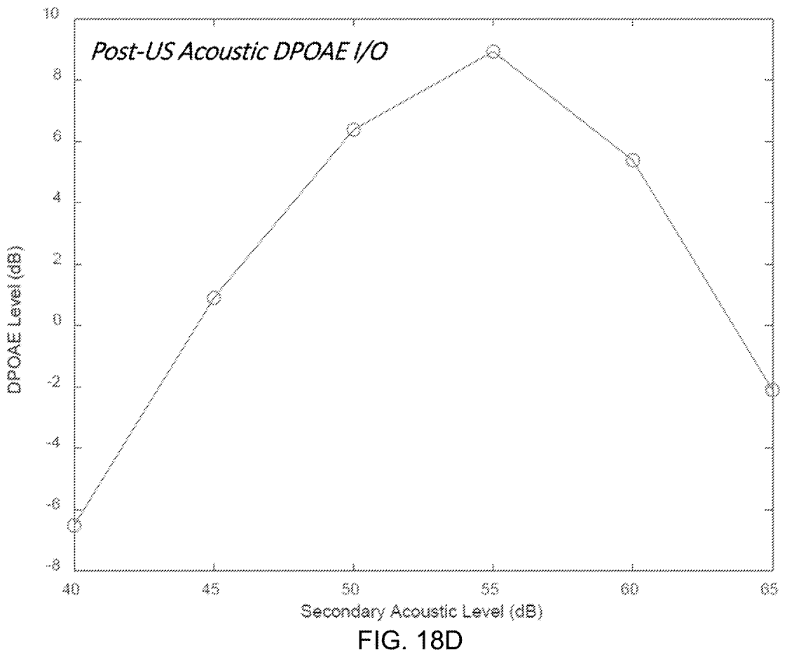

FIGS. 18A-18D show DPOAE input/output experimental results over a range of ultrasound amplitudes, demonstrating DPOAE recovery after exposure to ultrasound. FIG. 18A demonstrates a baseline measure of acoustic evoked DPOAEs prior to ultrasound exposure. FIG. 18B shows DPOAE levels swept over various ultrasound amplitudes for a bimodal experimental setup. FIG. 18C shows DPOAE levels swept over various ultrasound amplitudes for a dual transcranial ultrasound setup. After exposure to transcranial ultrasound, acoustic evoked DPOAEs demonstrated robust recovery to pre-ultrasound levels, as shown in FIG. 18D.

### Example 4

### Introduction

Ultrasound activation of the auditory system was first reported in 1948 by Gavreau and subsequent studies have reported the perception of ultrasonic sound when the ultrasound transmitter is coupled to the head of the patient. Previous work has also demonstrated that amplitude-modulating ultrasound can induce frequency-specific activation and pitch perception. Several studies have attributed this activation mechanism to bone-conduction, but recent work has demonstrated that ultrasound activates the auditory system via a fluid pathway. Ultrasound energy travels across the skull inducing vibrations in the cerebrospinal fluid (CSF). Since the CSF is continuous with the inner ear fluids due to the cochlear and vestibular aqueducts, the fluid vibrations travel into the inner ear and activate the auditory system. In this work, we aim at investigating the differences between a bone-conduction pathway and a fluid-conduction pathway by testing activation patterns from ultrasound and a bone conduction device. We also explore how more complex spatio-temporal stimuli activate the auditory system, whether presented via an air-driven, fluid-based, or bone-based pathway.

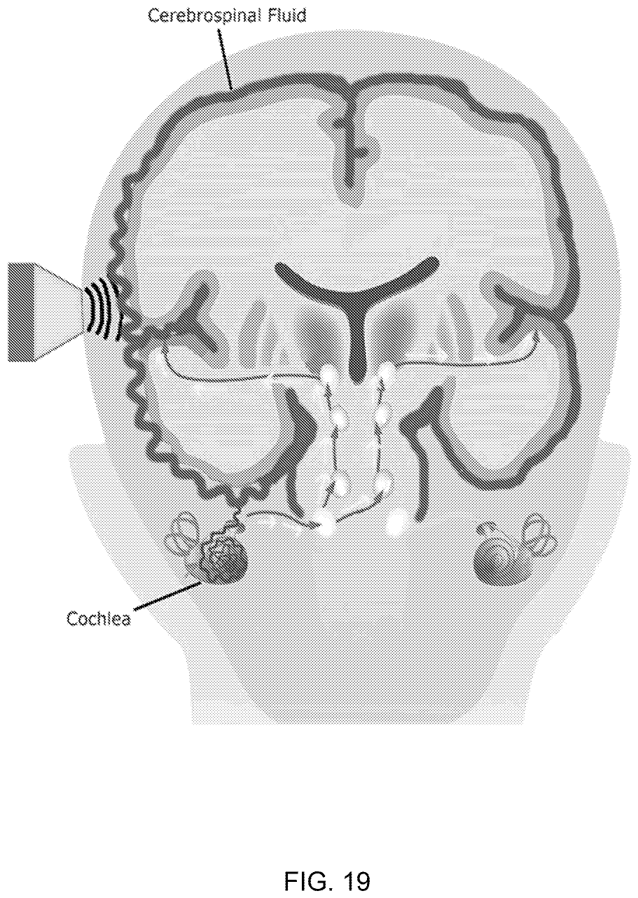

FIG. 19 shows a representation of the fluid pathway by which ultrasound activates the auditory system. US induces vibrations in the cerebrospinal fluid, which travel via the cochlear and vestibular aqueducts into the cochlea, activating the auditory system.

### Methods

Surgical prep: We implanted 2-shank, 32 site electrode arrays (NeuroNexus Technologies) along the tonotopic axis of the central nucleus of the inferior colliculus (ICC) of ketamine anesthetized guinea pigs (400-600 g). On the contralateral side, we performed a craniotomy in which an ultrasound transducer (Sonic Concepts) in a degassed water-filled focusing cone was coupled with agarose (center frequency 220 kHz, n=4). Rostral of this craniotomy, we coupled a bone conduction device (B-81 RadioEar, n=1) using a nut-screw system, in which the nut was cemented to the skull and the screw to the flat face of the B-81.

Stimulation: We collected frequency response maps (FRMs) by presenting pure tones (50 ms, 1 ms ramp on/off, 1-40 kHz, 8 steps/octave, 0-70 dB-SPL, 10 dB steps). We also modulated the ultrasound waveform with these tones to create ultrasound-FRMs (uFRMs) (50 ms, 1 ms ramp on/off, 7-70 kPa, 1-40 kHz, 8 steps/octave, 220 kHz carrier). We presented 11 guinea pig vocalizations via a speaker at 70 dB SPL (air-driven), as well as via the bone-conduction device (B-81 RadioEar, same power levels as the speaker) and ultrasound transducer (200 kPa). Stimuli were each presented 100 times to generate peri-stimulus time histograms (PSTHs). For the ultrasound signal, the envelopes of the vocalizations were extracted and used to modulate the ultrasound signal.

Analysis: Since we wanted to identify whether the evoked spike patterns had similarities in the spatio-temporal profile, we compared the PSTHs evoked by the same stimuli but different stimulation modalities using Earth mover's distance (EMD), also known as the Wasserstein metric.

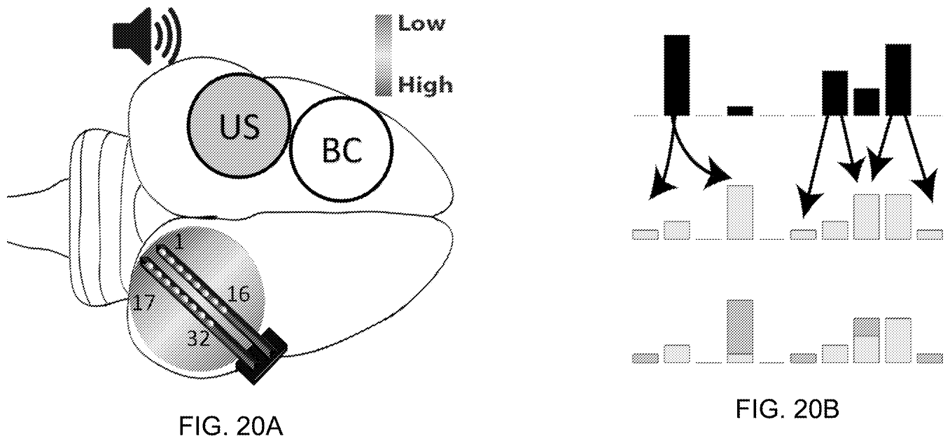

FIG. 20A shows a schematic demonstrating experimental set up. Electrodes are implanted in the ICC, with air-driven stimuli on the contralateral ear. The US transducer and bone conduction device were implanted contralaterally to the recording electrode.

FIG. 20B shows an EMD representation used to measure of the distance between two normalized histograms. The distance quantifies the amount of “earth” of one PSTH (black) necessary to fill in the “holes” of the second PSTH (green). The total cost of density movement is the EMD (grey). For both cases, a smaller value represents higher degree of similarity in the PSTHs.

### Results

### Frequency Response Maps

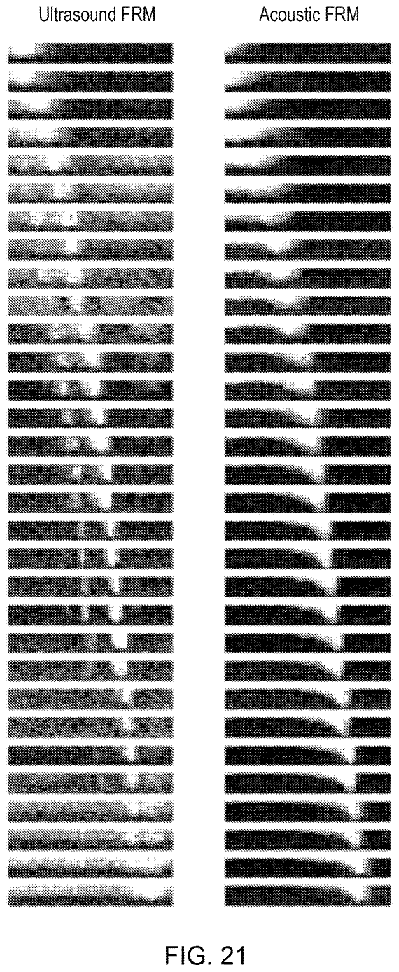

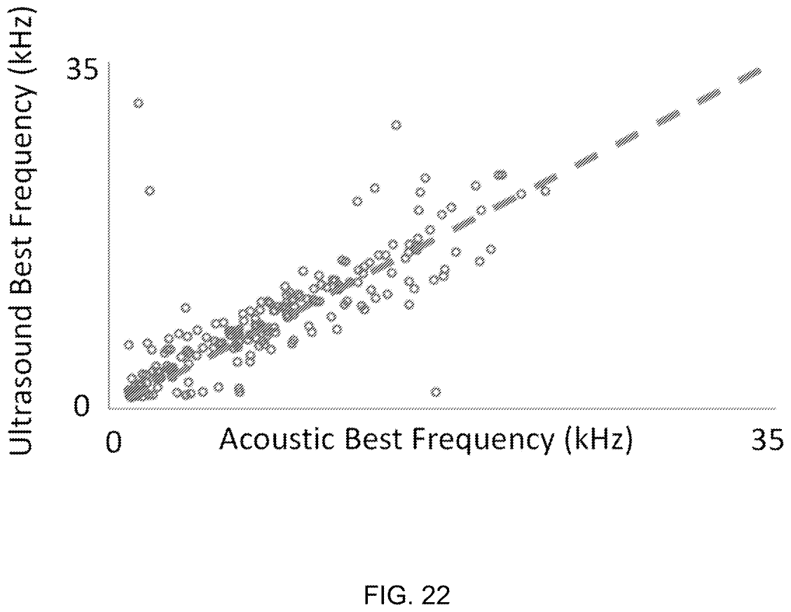

The tonotopic organization of the ICC is easily noticed in the frequency response maps shown in FIG. 21, regardless of the stimulation modality (ultrasound or acoustic). Channels have been sorted from lowest best frequency to highest best frequency (i.e., channels from both shanks are interleaved). FIG. 22 shows a plot of US best frequency against the acoustic best frequency per channel. The values lie on the y=x line demonstrating that the auditory system detects the modulation frequency of ultrasound. Interestingly, in the middle frequency range the ultrasound stimulation FRMs show a “sub-harmonic” response. These sub-harmonic responses are likely due to signal distortion caused by the nonlinear transmission/demodulation of the ultrasound waveform through the head to the cochlea. Another interesting finding lies in how the high frequency channels exhibit broad responses, even when modulated with lower frequencies. Although at the high-frequencies of modulation there's a noticed increase in activity, there is not as clear of a shift in best frequency response at those higher frequencies.

### Broad Population Responses

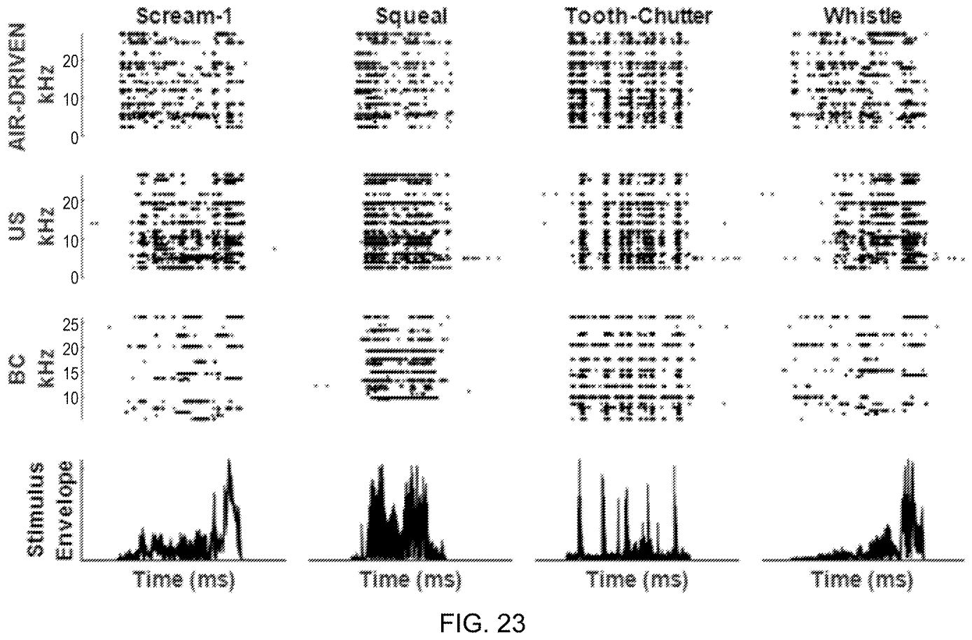

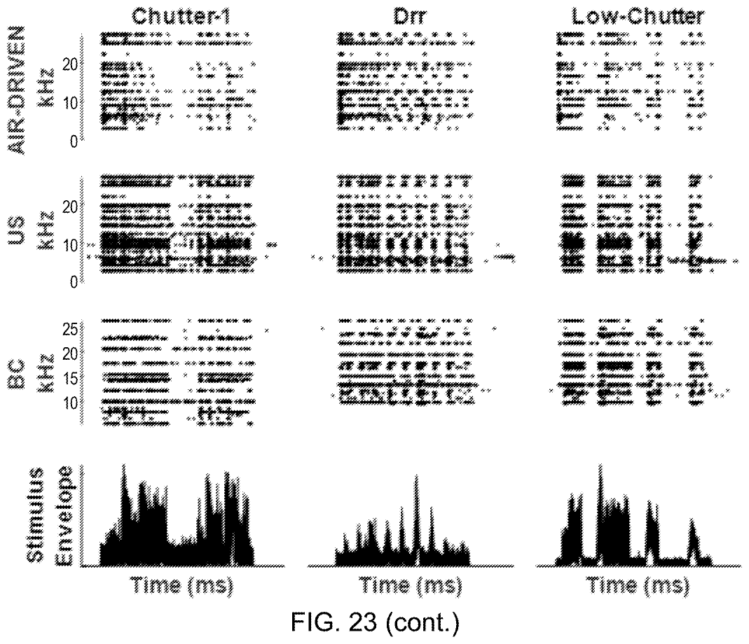

FIG. 23 shows population response maps evoked by different stimulation types (rows) and different stimulation signals (columns). The PSTHs across all channels were clipped at a set threshold, and time bins above this threshold were marked as responses while time bins below this threshold were removed. The air-driven responses were clipped at 20 spikes/time bin, while ultrasound and bone-conducted responses were both clipped at 10 spikes/time bin. Different threshold criteria were used across response type due to the increased activity evoked by the air-driven sounds. The bottom row shows the envelope of the stimulation signal.

All three modalities (US, BC, air-driven) evoke activity that generally follow the envelope of the stimulation signal across multiple frequency bands. Stimuli with rapid onset/offset behaviors (like ToothChutter or LowChutter) show a clear onset/offset response across all frequency bands. Surprisingly, Scream1 and Whistle don't appear to be well encoded in the population responses to bone conduction (BC), whereas in response to ultrasound, the envelope of the signals appears to be well described. It is encouraging to observe differences in US versus BC responses, supporting that US is activating the auditory system in a different manner than BC and more temporal specific manner than BC.

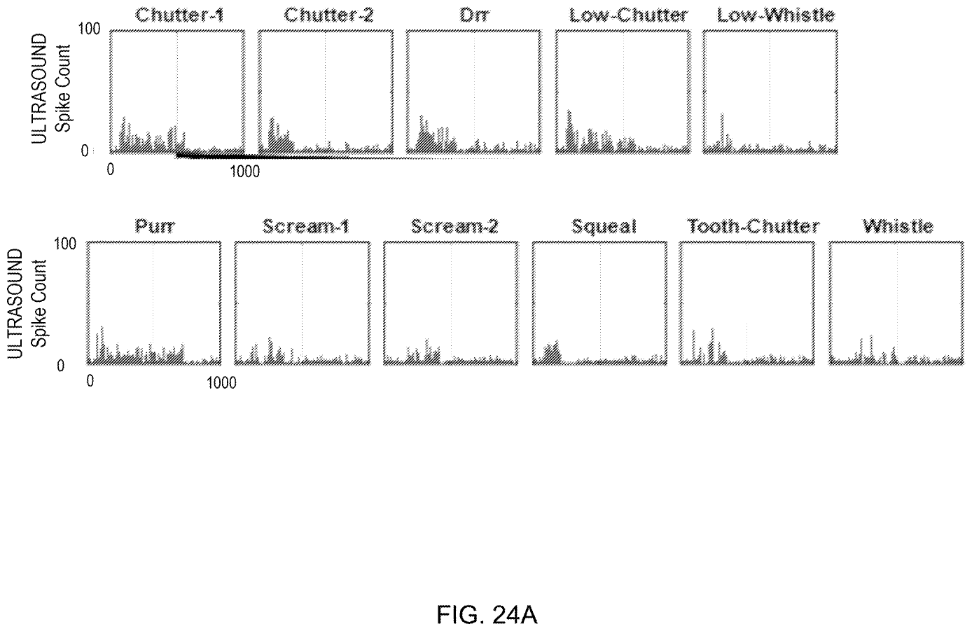

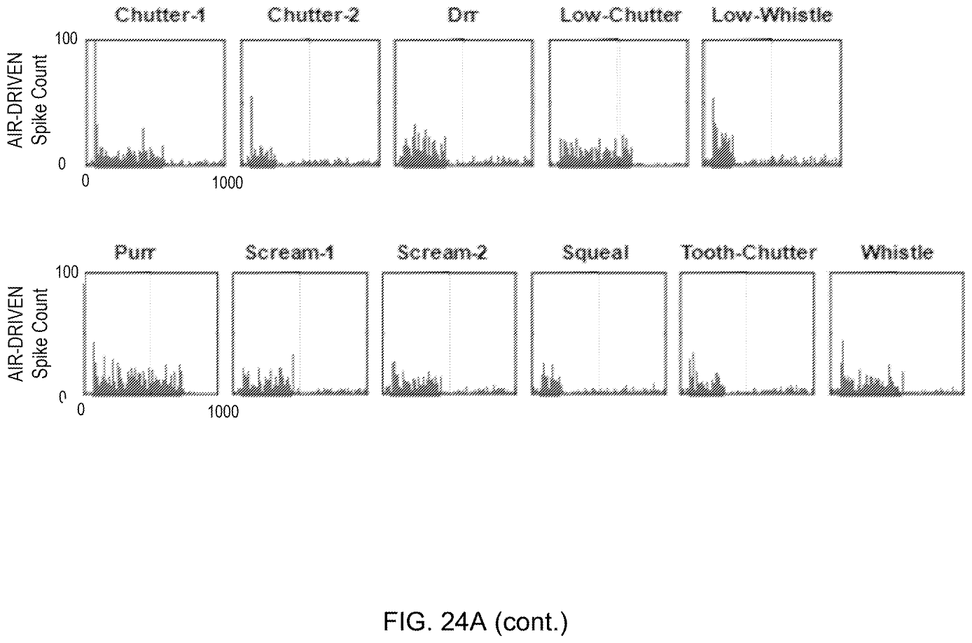

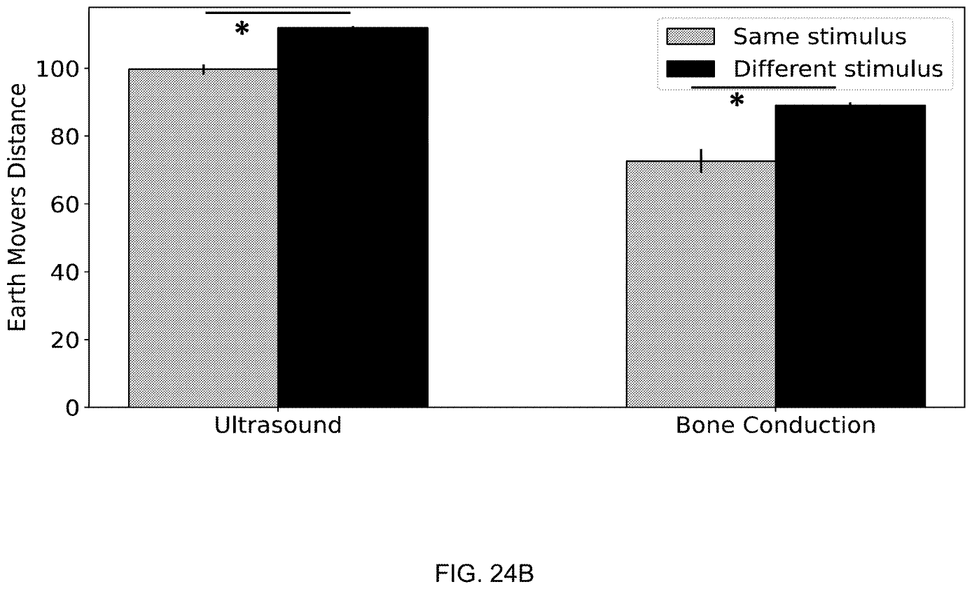

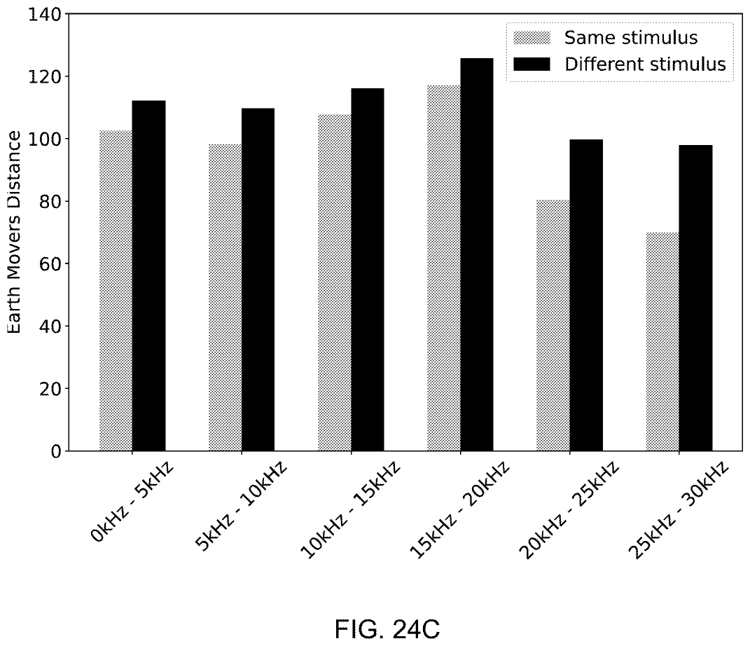

FIG. 24A shows how the Earth Mover's Distance (EMD) is estimated. In short, we estimated the EMD of the evoked responses between different modalities (US vs acoustic, or BC vs acoustic) by the same stimulus (i.e., Whistle-1 vs Whistle-1) or different stimuli (i.e., Scream-1 vs Purr). EMDs for all channels of same stimulus comparison were grouped, as well as those for different stimulus comparison. The averages of these grouped EMDs are plotted in FIG. 24B. A larger distance represents a smaller similarity between PSTHs. The distance between the same stimuli is lower than the distance between differing stimuli, showing there are similar spatio-temporal patterns encoded by the ultrasound stimulus and the air-driven stimulus. BC also has an improved performance even when comparing differing stimuli. BC may strongly encode certain frequency bands (regardless of stimuli) and so the distance across differing stimuli is smaller due to these consistencies. EMD highlights the similarity between same stimulus responses, showing that at the local level, PSTHs contain similar temporal patterns.

In FIG. 24C, instead of grouping all channels together, channels were grouped based on their acoustic best frequency to identify whether the similarity effects were frequency-specific. Only ultrasound vs acoustic is shown for easier understanding. As seen, the similarity in the spatio-temporal patterns (as estimated by EMD) is higher in the high frequency channels compared to other frequency bins. This is consistent whether the comparison is between similar stimuli or differing stimuli. Since we have noticed that ultrasound evokes broader activity in the high-frequency channels, the EMD is most likely capturing these effects across all stimuli. With more sophisticated US and encoding algorithms. Such effects can be reduced.

### Conclusions

Amplitude-modulated ultrasound can encode acoustic information, as quantified by responses recorded in the ICC. The analyses provided herein demonstrate that at a local level, we can observe similarities and many differences in the PSTH responses across all tested stimulation modalities, which supports complementary information can be provided by US stimulation compared to traditional sound or bone conduction methods.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. An apparatus for noninvasive assessment of at least one of hearing or intracranial pressure, comprising:
an ultrasound transducer configured to contact a head or a body of a subject;
a microphone configured to measure an auditory response; and
a controller operatively coupled to the ultrasound transducer and the microphone and configured to:
transmit an ultrasound wave pattern using the ultrasound transducer,
measure an auditory response using the microphone, and
analyze the auditory response to estimate at least one of hearing function or intracranial pressure of the subject.

2. The apparatus of claim 1, further comprising a sound generator, and wherein the controller is operatively coupled to the sound generator and configured to produce a sound signal using the sound generator.

3. The apparatus of claim 1, further comprising one or more electrodes operatively coupled to the controller and configured to be placed on the head of the subject to measure a neural response, wherein the controller is further configured to obtain a signal from the one or more electrodes indicative of the neural response of the subject.

4. The apparatus of claim 1, wherein the apparatus further comprises an optical imaging device operatively coupled to the controller and configured to acquire one or more images of at least one of a first eardrum of the subject, a second eardrum of the subject, a first eyeball of the subject, or a second eyeball of the subject.

5. The apparatus of claim 4, wherein the controller is further configured to assess the one or more images to characterize a vibration of at least one of the first eardrum of the subject, the second eardrum of the subject, the first eyeball of the subject, or the second eyeball of the subject.

6. The apparatus of claim 1, wherein the controller is further configured to assess at least one of hearing function or intracranial pressure based on the auditory response and a model that relates at least one of hearing function or intracranial pressure to the auditory response.

7. The apparatus of claim 3, wherein the controller is further configured to assess at least one of hearing function or intracranial pressure based on the auditory response, the neural response, and a model that relates intracranial pressure to the auditory response and the neural response.

8. The apparatus of claim 4, wherein the controller is further configured to assess at least one of hearing function or intracranial pressure based on the auditory response, the one or more images, and a model that relates intracranial pressure to the auditory response and the one or more images.

9. The apparatus of claim 4, wherein the controller is further configured to assess at least one of hearing function or intracranial pressure based on the auditory response, the one or more images, and the neural response in conjunction with a model that relates at least one of hearing function or intracranial pressure to the auditory response, the one or more images, and the neural response.

10-11. (canceled)

12. The apparatus of claim 1, wherein the auditory response comprises otoacoustic emissions, and
wherein the otoacoustic emissions comprise distortion production otoacoustic emissions.

13. (canceled)

14. The apparatus of claim 1, further comprising at least one of:
a pressure transducer configured to measure an intracranial pressure in the subject, or
a pressure modulator configured to modulate an intracranial pressure in the subject.

15. (canceled)

16. A method for noninvasive assessment of at least one of hearing or intracranial pressure, comprising:
providing an apparatus comprising:
an ultrasound transducer configured to contact a head or a body of a subject,
a microphone configured to measure an auditory response, and
a controller operatively coupled to the ultrasound transducer and the microphone;
transmitting, using the ultrasound transducer, an ultrasound wave pattern;
measuring, using the microphone, an auditory response; and
analyzing the auditory response to estimate at least one of hearing function or intracranial pressure of the subject.

17. The method of claim 16, wherein the apparatus further comprises a sound generator operatively coupled to the controller, and wherein the method further comprises producing a sound signal using the sound generator.

18. The method of claim 17, wherein the sound signal introduces acoustic stimulation to the subject and the ultrasound wave pattern introduces ultrasound stimulation to the subject.

19. The method of claim 18, wherein the acoustic and ultrasound stimulation are introduced simultaneously to the subject.

20. The method of claim 16, wherein analyzing the auditory response comprises:
comparing the auditory response to a database that comprises previously measured auditory responses that correspond with directly measured intracranial pressure values obtained in a plurality of subjects.

21. The method of claim 16, the method further comprising:
accessing a model trained on at least one of paired intracranial pressure data and auditory response data or paired hearing function data and auditory response data, and wherein analyzing the auditory response to estimate at least one of hearing function or intracranial pressure of the subject comprises analyzing the auditory response based on the model.

22. The method of claim 16, wherein the apparatus further comprises an optical imaging device operatively coupled to the controller, and wherein the method further comprises:
recording one or more optical images of at least one of a first eardrum of the subject, a second eardrum of the subject, a first eyeball of the subject, or a second eyeball of the subject; and
analyzing the auditory response and the one or more optical images to estimate at least one of hearing function or intracranial pressure of the subject.

23. The method of claim 16, wherein the apparatus further comprises one or more electrodes operatively coupled to the controller, and wherein the method further comprises:
recording a neural response using the one or more electrodes; and
analyzing the auditory response and the neural response to estimate at least one of hearing function or intracranial pressure of the subject.

24. The method of claim 16, wherein the apparatus further comprises one or more electrodes operatively coupled to the controller and an optical imaging device operatively coupled to the controller, and wherein the method further comprises:
recording a neural response using the one or more electrode;
recording one or more optical images of at least one of a first eardrum of the subject, a second eardrum of the subject, a first eyeball of the subject, or a second eyeball of the subject; and
analyzing at least one of the auditory response, the neural response, or the one or more optical images to estimate at least one of hearing function or intracranial pressure of the subject.

25. A method for generating a model for noninvasive assessment of intracranial pressure, comprising:
providing an apparatus comprising:
an ultrasound transducer configured to contact a head or a body of a subject,
a microphone configured to measure an auditory response,
a pressure transducer configured to measure intracranial pressure, and
a controller operatively coupled to the ultrasound transducer, the pressure transducer, and the microphone;
transmitting, using the ultrasound transducer, an ultrasound wave pattern to a plurality of subjects;
measuring, using the microphone, an auditory response from the plurality of subjects;
measuring, using the pressure transducer, an intracranial pressure from the plurality of subject, the intracranial pressure being paired to the auditory response;
calibrating a model for noninvasive assessment of intracranial pressure based on the paired intracranial pressures and auditory responses measured for the plurality of subject.
